(19) United States
(12) Patent Application Publication
(10) Pub. No.: US 2003/0073135 A1
(43) Pub. Date: Apr. 17, 2003

(54) Methods for improving a photosynthetic carbon fixation enzyme

(75) Inventor: Genhai Zhu, San Jose, CA (US)

Correspondence Address: MAXYGEN, INC., INTELLECTUAL PROPERTY DEPARTMENT, 515 GALVESTON DRIVE, RED WOOD CITY, CA, 94063, US

(73) Assignee: Maxygen, Inc.

(21) Appl. No.: 10/271,019

(22) Filed: Oct. 15, 2002

Related U.S. Application Data
(60) Provisional application No. 60/328,871, filed on Oct. 12, 2001.

(51) Int. Cl. G01N 33/53; C12N 9/02; A01H 1/00; C12N 15/82; C12N 15/74; C12N 1/21

(52) U.S. Cl. 435/7.1; 435/472; 435/189; 435/252.3; 800/284

(57) ABSTRACT

The invention relates to methods and compositions for generating, modifying, adapting, and optimizing polynucleotide sequences that encode proteins having photosynthetic carbon fixation activities, including Rubisco and Rubisco activase activities, which are useful for introduction into plant species, agronomically-important microorganisms, and other hosts, and related aspects.

### CROSS REFERENCE TO RELATED APPLICATIONS

This application claims priority to U.S. Ser. No. 60/328,871, filed Oct. 12, 2001, the disclosure of which is incorporated by reference for all purposes.

### FIELD OF THE INVENTION

The invention relates to methods and compositions for generating, modifying, adapting, and optimizing polynucleotide sequences that encode proteins having photosynthetic carbon fixation activities, including Rubisco and Rubisco activase activities, which are useful for introduction into plant species, agronomically-important microorganisms, and other hosts, and related aspects.

### BACKGROUND

Ribulose-1,5-bisphosphate carboxylase/oxygenase (Rubisco, E. C. 4.1.1.39) is the most abundant and perhaps most important enzyme on earth. It catalyzes the first and rate-limiting step in photosynthetic carbon fixation, the transfer of atmospheric CO₂ to ribulose-1,5-bisphosphate. As such, it is the only known enzyme able to remove CO₂ from the atmosphere. Because of its keystone position in biomass production, the importance of Rubisco to agriculture is hard to overstate. Cash receipts for American agricultural products in 1997 were $209 billion, of which $112 billion were earned directly from crops (Economic Research Service, USDA). Thus, any incremental increase in crop productivity will be leveraged through a huge sector of the U.S. agricultural economy. For several reasons, it is widely supposed that increasing Rubisco's catalytic efficiency will result in a significant increase in plant productivity. First, the reaction catalyzed by Rubisco is rate limiting to plant growth under optimum growing conditions (high temperature and light intensity, abundant nitrogen). Second, compared to many other enzymes, Rubisco seems to be an inefficient catalyst that leaves a great deal of room to be optimized.

As a catalyst, Rubisco appears to be sub-optimal in three respects. First, its catalytic cycling rate (k_{cat}) at about 3 reactions per second, for the enzymes from higher plants, is relatively slow. To compensate for its low activity, plants deposit large amounts of Rubisco enzyme in their green tissues. Indeed, Rubisco accounts for more than 35% of leaf total soluble proteins. As elaborated in Section II, increasing Rubisco's catalytic efficiency would proportionally increase the rate of photosynthesis and, in turn, increase plant productivity. Second, Rubisco cannot effectively distinguish CO₂ from O₂ and, consequently, it catalyzes an oxygenation reaction that leads to the loss of approximately 25% to 40% of fixed carbon (FIG. 2). Theoretically, it is possible to increase plant productivity up to 50% by reducing or eliminating Rubisco's oxygenase activity. Third, Rubisco is activated by Rubisco activase, Rubisco activase is notoriously heat labile and inactivation of Rubisco activase under hot growing conditions is correlated with loss of photosynthetic carbon fixation.

Rubisco has become one of the most intensively investigated plant enzymes. Evolution and adaptation of Rubisco in its various native hosts have resulted in a naturally occurring diversity of enzymatic properties (Jordan and Ogren, 1981). Compared to plant Rubisco, the enzyme from prokaryotic photosynthetic bacteria generally possesses higher catalytic actitivty (k_{cat}8−16 s⁻¹), but low CO₂/O₂ selectivity (τ≈13−40). τ is the ratio of k_{cat(carboxylation)/}K_{m(CO2)} over k_{cat(oxygenation)}/K_{m(O2)} (Laing et al., 1974). Rubisco from higher plants including crop species exhibits low k_{cat} (≈3 s⁻¹), and an intermediate CO₂/O₂ selectivity (τ≈80). The recently-assayed Rubisco from red algae shows the highest CO₂/O₂ selectivity yet measured (τ≈140−300, Ezaki et al., 1999; Read and Tabita, 1994; Uemura et al., 1997), but the k_{cat} assayed at 25° C. is lower than that of higher plant Rubisco. This diversity among Rubisco enzymes stimulated research aimed at understanding the structure/function relationships that account for the variation of the catalytic parameters k_{cat} and τ. Engineering a better Rubisco through knowledge of the structural determinants of k_{cat} and τ constitutes the so called “rational approach.”

Rubisco from different organisms displays different physical and chemical features. Its holoenzyme is a multi-subunit complex. The primitive form is a large/large subunit dimer (L₂). The L₂ enzyme is mainly present in anaerobic proteobacteria, but the L₂ enzyme is also formed in some eukaryotic algae under anaerobic conditions. In all higher plants and cyanobacteria, Rubisco is composed of eight large (L) and eight small (S) subunits (L₈S₈). The L subunit is encoded by a chloroplast gene (rbcL), and the S subunit is encoded by a nuclear gene family (rbcS). So far, only L₂, the cyanobacterial L₈S₈ enzyme, and an L₈ enzyme from a hyperthermophilic alga have been expressed and assembled in E. coli. Expression of higher plant Rubisco L and S simultaneously in E. coli resulted in no holoenzyme being formed. Consequently, most Rubisco engineering research has been limited to prokaryotic enzymes.

For more than 20 years a number of researchers have attempted to improve Rubisco, using a variety of approaches. See, e.g., Mann, C. C., Science, 283, 314-316, 1999, and references cited therein. Indeed, the quest for a better Rubisco has been called a “Holy Grail” of plant biology. To date, there has been little success in the creation of an improved Rubisco. Recombination based methods for producing a modified Rubisco enzyme having increased catalytic efficiency and selectivity for CO₂ are described in U.S. patent application No. 09/437,726.

Rubisco activase is a nuclear encoded enzyme that is a major determinant of the fraction of Rubisco that is catalytically active (R. G. Jensen (2000) PNAS 97:12937-38). Rubisco activase, through some mechanism involving ATP hydrolysis and highly specific interaction with Rubisco, loosens the binding of inhibitory sugar phosphates to Rubisco and activates the enzyme. It is well documented that crop photosynthesis is inhibited by moderately elevated temperature. Photosynthetic CO₂ fixation declines when temperature exceeds 30° C. for wheat and 35° C. for cotton. The decline in photosynthesis under high temperature and saturated light in crops can occur without a decrease in stomatal conductance and CO₂ influx. Since plants grow in a fluctuating environmental temperature, such a decline of photosynthesis constrains the potential of the plant productivity. Recent research showed that Rubisco activase is particularly thermo-labile. See, e.g., Crafts-Bandner SJ and Salvucci ME, PNAS 97, 13430-13435, 2000; Salvucci et al., Plant Physiology 127, 1053-64, 2001; and Crafts-Bandner SJ and Salvucci ME, Plant Physiology 129, 1773-1780, 2002. It is thought that loss of Rubisco activase activity at elevated temperature is a primary cause for the loss of Rubisco activation, which in turn reduces photosynthetic CO₂ fixation. In addition, a mathematical model indicates that Rubisco activase limits non-steady-state photosynthesis in plants receiving fluctuating light (e.g., light flecks formed in a canopy), even at moderate temperatures (Mott KA and Woodrow IE, J Exp Botany 51, 399-406, 2000).

An obstacle hindering the improvement of Rubisco is the deficiencies in currently available host systems for the expression and assembly of functional higher plant Rubisco. In screening a large number of variants for enhanced activity, preferred host systems have included E. coli, yeast, cyanobacteria and green algae In the case of prokaryotic Rubisco, the large subunit (i.e., the L₈ core) of prokaryotic Rubisco is soluble, and catalytically competent holoenzyme can be formed in E. coli with the help of a chaperone protein (GroEL) present in E. coli. In contrast, the large subunit from higher plant Rubisco is insoluble; this is thought to be caused by a hydrophobic surface that is protected by the small subunit in the holoenzyme. In chloroplasts, assembly of the large subunits with mature small subunits is mediated by a chaperone protein, Rubisco binding protein (cpn60). The chaperone protein is believed to prevent improper aggregation of large subunits by protecting exposed hydrophobic surfaces during the last stages of the folding or assembly process. Co-expression of large and small subunits in E. coli results in no active holoenzyme being formed, suggesting that inappropriate folding of the large subunit may have occurred before the small subunit was able to bind. The difficulty in expressing higher plant Rubisco in a suitable host has made it difficult to engineer improved variants of the enzyme.

Thus, there exists a need for improved methods for producing plants and agricultural photosynthetic microbes with improved variants of enzymes involved in carbon fixation, for example Rubisco and Rubisco activase. There also exists a need for improved host systems for expressing these enzymes. The present invention meets these and other needs and provides such improvements and opportunities.

The references discussed herein are provided solely for their disclosure prior to the filing date of the present application. Nothing herein is to be construed as an admission that the inventors are not entitled to antedate such disclosure by virtue of prior invention. All publications cited are incorporated herein by reference, whether specifically noted as such or not.

### SUMMARY OF THE INVENTION

In a broad general aspect, the present invention provides a method for the rapid evolution of a polynucleotide sequences or sequences encoding a photosynthetic carbon fixation protein. As used herein, the term “photosynthetic carbon fixation protein” refers to any protein that is involved in the biological process of photosynthetic carbon fixation. Often these proteins function as enzymes. The proteins can be of prokaryotic or eukaryotic origin, and in preferred embodiments are modified variants that have not been previously observed in nature. In preferred embodiments of the invention, “photosynthetic carbon fixation protein” refers to Rubisco or Rubisco activase, including subunits thereof (e.g., both regulatory subunit (small subunit, S; gene designation, rbcS) and catalytic subunit (large subunit, L; gene designation, rbcL), respectively, as appropriate for Form I (L₈S₈) and Form II (L₂) Rubisco). The term “photosynthetic carbon fixation polynucleotide” refers to a polynucleotide that encodes a photosynthetic carbon fixation protein, or a fragment of such a polynucleotide which can be used in some aspects of the invention as described below.

In general, polynucleotide sequence recombination (e.g., shuffling) and phenotype selection, such as detection of a parameter of enzyme activity, is employed recursively to generate polynucleotide sequences which encode novel proteins having desirable enzymatic catalytic function(s), regulatory function(s), and related enzymatic and physicochemical properties. Although the method is believed broadly applicable to evolving enzymes involved in photosynthesis or carbon fixation having desired properties, the invention is described principally with reference to the metabolic enzyme activities of plants and/or photosynthetic microbes defined as Rubisco and Rubisco activase, and any subunits thereof.

The invention provides an isolated polynucleotide encoding an enhanced photosynthetic carbon fixation protein having an improved phenotype. Examples of improved phenotypes include increased catalytic activity, increased thermal stability, and/or increased CO₂/O₂ selectivity catalytic activity relative to a protein encoded by a parental polynucleotide encoding a naturally-occurring photosynthetic carbon fixation protein enzyme.

In one aspect, the invention provides an isolated polynucleotide encoding an enhanced Rubisco protein having Rubisco catalytic activity wherein τ is significantly greater than a protein encoded by a parental polynucleotide encoding a naturallyoccurring Rubisco enzyme, e.g., greater to a degree that is statistically significant. This can be accomplished, for example, by lowering the Km for CO₂. Typically, the Km for CO₂ will be at least one-half logarithm unit lower than the parental sequence, preferably the Km will be at least one logarithm unit lower, and desirably the Km will be at least two logarithm units lower, or more. The isolated polynucleotide encoding an enhanced Rubisco protein and in an expressible form can be transferred into a host plant, such as a crop species, wherein suitable expression of the polynucleotide in the host plant results in improved carbon fixation efficiency as compared to the naturally-occurring host plant species, usually under certain atmospheric conditions. The isolated polynucleotide can encode a single subunit Rubisco, such as a Form II bacterial form, or may encode a large (L) subunit or small (S) subunit of a multisubunit Form I Rubisco such as that found in cynaobacteria, green algae, and higher plants. The isolated polynucleotide can comprise a substantially full-length or full-length coding sequence substantially identical to a naturally occurring rbcS gene and/or an rbcL gene, typically comprising a shuffled rbcL gene or a shuffled rbcL gene, or both.

The invention further provides an isolated polynucleotide encoding an enhanced Rubisco protein having Rubisco catalytic activity wherein the catalytic activity, which can be defined in terms of kcat or kcat/Km for carboxylation, is significantly greater than a protein encoded by a parental polynucleotide encoding a naturallyoccurring Rubisco enzyme.

The invention provides an isolated polynucleotide encoding an enhanced Rubisco activase protein having Rubisco activase catalytic activity that is significantly greater than a protein encoded by a parental polynucleotide encoding a naturally-occurring Rubisco activase enzyme, e.g., greater to a degree that is statistically significant. In an embodiment of the invention, the kcat of the ATPase activity of the enhanced Rubisco activase is at least 2-fold greater (and still more preferably 5, 10 or 100-fold greater) than that of a protein encoded by a parental polynucleotide encoding a naturally-occurring Rubisco activase enzyme. In an embodiment of the invention, the temperature dependence of the enzymes catalytic activity is shifted to a higher temperature or improved at higher temperatures. For example, an enhanced Rubisco activase protein can have improved catalytic activity at temperatures exceeding 25° C., 30° C., 35° C. or 40° C.

The invention further provides an isolated polynucleotide encoding an enhanced Rubisco activase protein having a thermal stability that is significantly greater than a protein encoded by a parental polynucleotide encoding a naturallyoccurring Rubisco activase enzyme. In an embodiment of the invention, the temperature at which 50% of the ATPase activity of enhanced Rubisco activase is lost in a 1 hr incubation is significantly greater than for a protein encoded by a parental polynucleotide, preferably 5° C. greater and more preferably 10° C. greater. In a further embodiment of the invention, the temperature at which the an enhanced Rubisco activase loses its native tertiary structure is significantly greater than for a protein encoded by a parental polynucleotide, preferably 5° C. greater and more preferably 10° C. greater. Loss of native tertiary structure can be determined by any of a number of methods known in the art, e.g., by change in light scattering or aggregation with Rhodanese (Salvucci et al. (2002)).

In another aspect of the invention, an enhanced Rubisco activase is introduced into a plant, thereby providing a transgenic plant with an increased temperature optimum for photosynthesis. In preferred embodiments of the invention the temperature optimum for photosynthesis is increased by at least 5° C., or in sometimes at least 10° C., relative to the corresponding plant that has not been modified by the introduction of the ability to express the enhanced Rubisco activase.

The invention further provides a eukaryotic host system for the expression of a photosynthetic carbon fixation protein. In a preferred embodiment, the photosynthetic carbon fixation protein is Rubisco or a variant thereof, and the host system is a species of chlamydomonas, with chlamydomonas reindartii particularly preferred. In another embodiment, the photosynthetic carbon fixation protein is Rubisco activase or a variant thereof, and the host system is a species of chlamydomonas, with chlamydomonas reindartii particularly preferred.

In a variation, the invention provides an isolated polynucleotide encoding an enhanced Rubisco protein having Rubisco catalytic activity wherein the Km for O₂ is significantly higher than a protein encoded by a parental polynucleotide encoding a naturally-occurring Rubisco enzyme or subunit. In an aspect, the enhanced Rubisco protein is often a L subunit which is catalytically active in the presence of a complementing S subunit. In an aspect, the enhanced Rubisco protein is a L subunit which is catalytically active in the absence of a complementing S subunit, such as for example and not limitation a Rubisco L subunit which is at least 90 percent sequence identical to a naturally occurring Form II L subunit.

In a variation, the invention provides an isolated polynucleotide encoding an enhanced Rubisco protein having Rubisco catalytic activity wherein the ratio of the Km for CO₂ to the Km for O₂ is significantly lower than a protein encoded by a parental polynucleotide encoding a naturally-occurring Rubisco enzyme.

The invention provides an enhanced Rubisco protein having Rubisco catalytic activity wherein: (1) the Km for CO₂ is significantly lower than a protein encoded by a parental polynucleotide encoding a naturally-occurring Rubisco enzyme, (2) the Km for O₂ is significantly higher than a protein encoded by a parental polynucleotide encoding a naturally-occurring Rubisco enzyme, and/or (3) the ratio of the Km for CO₂ to the Km for O₂ is significantly lower than a protein encoded by a parental polynucleotide encoding a naturally-occurring Rubisco enzyme.

An improved Rubisco activase, or variant thereof, and a polynucleotide encoding the same are provided. In some embodiments, the polynucleotide is operably linked to a transcription regulation sequence forming an expression construct, which may be linked to a selectable marker gene. In some embodiments, such a polynucleotide is present as an integrated transgene in a plant chromosome, or more typically on a chloroplast chromosome in a format for expression and processing of the enzyme in chloroplasts, which may be accomplished by homologous recombination targeting into a chloroplast genome. It can be desirable for such a polynucleotide transgene to be transmissible via germline transmission in a plant; in the case of coding sequences transferred to chloroplasts, it is often accompanied by a selectable marker gene which affords a means to select for progeny which retain chloroplasts having the transferred improved Rubisco activase encoding sequence.

In an aspect, the invention provides a hybrid Rubisco activase composed of a shufflant comprising a sequence of at least 25 contiguous nucleotides at least 95 percent identical to a first Rubisco activase gene and a sequence of at least 25 contiguous nucleotides at least 95 percent identical to a second Rubisco activase gene, and a polynucleotide encoding same, and typically encoding a substantially fulllength Rubisco activase protein, usually comprising at least 90 percent of the coding sequence length, but not necessarily sequence identity, of a naturally occurring Rubisco activase protein. In some embodiments, the polynucleotide will be operably linked to a transcription regulation sequence forming an expression construct, which may be linked to a selectable marker gene. In some embodiments, such a polynucleotide is present as an integrated transgene in a plant chromosome. It can be desirable for such a polynucleotide transgene to be transmissible via germline transmission in a plant.

The invention provides expression constructs, including plant transgenes, wherein the expression construct comprises a transcriptional regulatory sequence functional in plants operably linked to a polynucleotide encoding an enhanced photosynthetic carbon fixation protein. With respect to polynucleotide sequences encoding Form I Rubisco L subunit proteins, it is generally desirable to express such encoding sequences in plastids, such as chloroplasts, for appropriate transcription, translation, and processing. The invention further provides plants and plant germplasm comprising said expression constructs, typically in stably integrated or other replicable form which segregates and can be stably maintained in the host organism, although in some embodiments it is desirable for commercial reasons that the expression sequence not be in the germline of sexually reproducible plants.

The invention provides a method for obtaining an isolated polynucleotide encoding an enhanced Rubisco activase protein increased thermal stability and/or increased catalytic activity, the method comprising: (1) recombining sequences of a plurality of parental polynucleotide species encoding at least one Rubsico activase sequence under conditions suitable for sequence shuffling to form a resultant library of sequence-shuffled Rubisco polynucleotides, (2) transferring said library into a plurality of host cells forming a library of transformants wherein sequence-shuffled Rubisco activase polynucleotides are expressed, (3) assaying individual or pooled transformants for thermal stability and/or catalytic activity, and (4) recovering the sequence-shuffled Rubisco activase polynucleotide from at least one enhanced transformant. Optionally, the recovered sequence-shuffled Rubisco activase polynucleotide encoding an enhanced Rubisco activase is recursively shuffled and selected by repeating steps 1 through 4, wherein the recovered sequence-shuffled Rubisco activase polynucleotide is used as at least one parental sequence for subsequent shuffling. Catalytic activity can be assayed by determining the rate of ATP hydrolysis, for example using the spectrophotometric methods described by Crafts-Brandner and Salvucci (2000). Temperature stability can be monitored using any of a number of methods that will be apparent to one of skill in the art, e.g., by determining the temperature response of Rubisco activation or ATP hydrolysis, or by monitoring loss of native protein structure by detecting changes in light scattering or aggregation with Rhodanese (Salvucci et al. (2001)).

In a variation, the sequence-shuffled photosynthetic carbon fixation polynucleotide operably linked to an expression sequence is also linked, in polynucleotide linkage, to an expression cassette encoding a selectable marker gene. Transformants are propagated on a selective medium to ensure that transformants which are assayed for catalytic activity and/or thermal stability contain a sequence-shuffled photosynthetic carbon fixation protein encoding sequence in expressible form. In embodiments wherein a polynucleotide encoding a Rubisco L subunit are to be introduced into host cells which possess chloroplasts, the L subunit encoding sequence is generally operably linked to a transcriptional regulatory sequence functional in chloroplasts and the resultant expression cassette is transferred into the host cell chloroplasts, such as by biolistics, polyethylene glycol (PEG) treatment of protoplasts, or an other suitable method.

Optionally, the host cell for transformation with sequence-shuffled polynucleotides encoding Rubisco is a Synechocystis mutant which lacks a Rubisco subunit protein, such as Synechocystis PCC6803, a mutant Rhodospirillum rubrum, or an equivalent. Another preferred host cell is Chlamydomonas particularly Chlamydomonas reindartii.

In an embodiment of the method, the host cell comprises a cell expressing a complementing subunit of Rubisco which is capable of interacting with a Rubisco protein encoded by sequence-shuffled polypeptides encoding a Rubisco subunit. For example, if the shuffled polynucleotides encode a large subunit of Rubisco, a host cell for the transformation may endogenously encode a small subunit of Rubisco that may interact with a functional large subunit encoded by the shuffled polynucleotides. It is often desirable that such host cells lack expression of the endogenous Rubisco subunit corresponding to (e.g., cognate to) the type of subunit encoded by the shuffled polynucleotides. Mutant cell lines are available in the art and novel mutant Rubisco-deficient cells can be obtained by selecting from a pool of mutagenized cells those mutants which have lost detectable Rubisco activity, or by homologous gene targeting of rbcL and/or rbcS genes.

The invention provides a plant cell protoplast and clonal progeny thereof containing a sequence-shuffled polynucleotide encoding a photosynthetic carbon fixation protein which is not encoded by the naturally occurring genome of the plant cell protoplast. The invention also provides a collection of plant cell protoplasts transformed with a library of sequence-shuffled photosynthetic carbon fixation polynucleotide in expressible form. The invention further provides a plant cell protoplast co-transformed with at least two species of library members wherein a first species of library members comprise sequence-shuffled Rubisco large subunit polynucleotides and a second species of library members comprise sequence-shuffled Rubisco small subunit polynucleotides. Typically, the large subunit polynucleotides are transferred into a plastid compartment for expression and processing, such as by transfer into chloroplasts in a format suitable for expression in the plastid, such as for example and not limitation as a recombinogenic construct for general targeted recombination into a chloroplast chromosome. Typically, small subunit polynucleotides are transferred into the protoplast nucleus for expression, and, if desired, integration or homologous recombination (or gene replacement of the endogenous rbc gene(s)).

The invention also provides a regenerated plant containing at least one species of replicable or integrated polynucleotide comprising a sequence-shuffled portion and encoding a photosynthetic carbon fixation protein. The invention provides a method variation wherein at least one round of phenotype selection is performed on regenerated plants derived from protoplasts transformed with sequence-shuffled photosynthetic carbon fixation polynucleotide library members.

The invention provides methods for making Rubisco activase variants with improved phenotypes such as enhanced thermal stability and/or improved catalytic activity. The invention further provides Rubisco activase variants, polynucleotides encoding such variants, cells and organisms expressing these variants, including plants with enhanced carbon fixation activity, for example at high temperatures.

In another aspect, the invention provides eukaryotic expression systems capable of expressing heterologous carbon fixation enzymes. In a preferred embodiment, Rubisco variants are expressed in a eukaryotic host such as Chlamydomonas reindartii, thereby facilitating genetic manipulation and screening of the enzyme.

Other features and advantages of the invention will be apparent from the following description of the drawings, preferred embodiments of the invention, the examples, and the claims.

### DETAILED DESCRIPTION

### Definitions

Unless defined otherwise, all technical and scientific terms used herein have the same meaning as commonly understood by one of ordinary skill in the art to which this invention pertains. Although methods and materials similar or equivalent to those described herein can be used in the practice or testing of the present invention, the preferred methods and materials are described. For purposes of the present invention, the following terms are defined below.

The term “shuffling” is used herein to indicate recombination between similar but non-identical polynucleotide sequences. Generally, more than one cycle of recombination is performed in DNA shuffling methods. In some embodiments, DNA shuffling may involve crossover via nonhomologous recombination, such as via cre/lox and/or flp/frt systems and the like, such that recombination need not require substantially homologous polynucleotide sequences. In silico and oligonucleotide mediated approaches also do not require similarity/homology. Homologous and non-homologous recombination formats can be used, and, in some embodiments, can generate molecular chimeras and/or molecular hybrids of substantially dissimilar sequences. Viral recombination systems, such as template-switching and the like can also be used to generate molecular chimeras and recombined genes, or portions thereof. A general description of shuffling is provided in WO98/13487 and WO98/13485, both of which are incorporated herein in their entirety by reference; in case of any conflicting description of definition between any of the incorporated documents and the text of this specification, the present specification provides the principal basis for guidance and disclosure of the present invention.

The term “related polynucleotides” means that regions or areas of the polynucleotides are identical and regions or areas of the polynucleotides are heterologous.

The term “chimeric polynucleotide” means that the polynucleotide comprises regions which are wild-type and regions which are mutated. It may also mean that the polynucleotide comprises wild-type regions from one polynucleotide and wild-type regions from another related polynucleotide.

The term “cleaving” means digesting the polynucleotide with enzymes or breaking the polynucleotide (e.g., by chemical or physical means), or generating partial length copies of a parent sequence(s) via partial PCR extension, PCR stuttering, differential fragment amplification, or other means of producing partial length copies of one or more parental sequences. A fragmented population of nucleic acids is produced by cleavage of a polynucleotide as indicated, or by producing oligonucleotide sets that correspond to one or more parental nucleic acid.

The term “population,” as used herein, means a collection of components such as polynucleotides, nucleic acid fragments, or proteins. A “mixed population” means a collection of components which belong to the same family of nucleic acids or proteins (i.e. are related) but which differ in their sequence (i.e. are not identical) and hence in their biological activity.

The term “mutations” means changes in the sequence of a parent nucleic acid sequence (e.g., a gene or a microbial genome, transferable element, or episome) or changes in the sequence of a parent polypeptide. Such mutations may be point mutations such as transitions or transversions. The mutations may be deletions, insertions or duplications.

The term “recursive sequence recombination” as used herein refers to a method whereby a population of polynucleotide sequences are recombined with each other by any suitable recombination means (e.g., sexual PCR, homologous recombination, site-specific recombination, shuffling, etc.) to generate a library of sequence-recombined species which is then screened or subjected to selection to obtain those sequence-recombined species having a desired property; the selected species are then subjected to at least one additional cycle of recombination with themselves and/or with other polynucleotide species and at subsequent selection or screening for the desired property.

The term “amplification” means that the number of copies of a nucleic acid fragment is increased.

The term “naturally-occurring” as used herein as applied to an object refers to the fact that an object can be found in nature. For example, a polypeptide or polynucleotide sequence that is present in an organism that can be isolated from a source in nature and which has not been intentionally modified by man in the laboratory is naturally-occurring. As used herein, laboratory strains and established cultivars of plants which may have been selectively bred according to classical genetics are considered naturally-occurring. As used herein, naturally-occurring polynucleotide and polypeptide sequences are those sequences, including natural variants thereof, which can be found in a source in nature, or which are sufficiently similar to known natural sequences that a skilled artisan would recognize that the sequence could have arisen by natural mutation and recombination processes.

As used herein “predetermined” means that the cell type, non-human animal, or virus may be selected at the discretion of the practitioner on the basis of a known phenotype.

As used herein, “linked” means in polynucleotide linkage (i.e., phosphodiester linkage). “Unlinked” means not linked to another polynucleotide sequence; hence, two sequences are unlinked if each sequence has a free 5′ terminus and a free 3′ terminus.

As used herein, the term “operably linked” refers to a linkage of polynucleotide elements in a functional relationship. A nucleic acid is “operably linked” when it is placed into a functional relationship with another nucleic acid sequence. For instance, a promoter or enhancer is operably linked to a coding sequence if it affects the transcription of the coding sequence. Operably linked means that the DNA sequences being linked are typically contiguous and, where necessary to join two protein coding regions, contiguous and in reading frame. However, since enhancers generally function when separated from the promoter by several kilobases and intronic sequences may be of variable lengths, some polynucleotide elements may be operably linked but not contiguous. A structural gene (e.g., a RUBISCO gene) which is operably linked to a polynucleotide sequence corresponding to a transcriptional regulatory sequence of an endogenous gene is generally expressed in substantially the same temporal and cell type-specific pattern as is the naturallyoccurring gene.

As used herein, the terms “expression cassette” refers to a polynucleotide comprising a promoter sequence and, optionally, an enhancer and/or silencer element(s), operably linked to a structural sequence, such as a cDNA sequence or genomic DNA sequence. In some embodiments, an expression cassette may also include polyadenylation site sequences to ensure polyadenylation of transcripts. When an expression cassette is transferred into a suitable host cell, the structural sequence is transcribed from the expression cassette promoter, and a translatable message is generated, either directly or following appropriate RNA splicing. Typically, an expression cassette comprises: (1) a promoter, such as a CaMV 35S promoter, a NOS promoter or a rbcS promoter, or other suitable promoter known in the art, (2) a cloned polynucleotide sequence, such as a cDNA or genomic fragment ligated to the promoter in sense orientation so that transcription from the promoter will produce a RNA that encodes a functional protein, and (3) a polyadenylation sequence. For example and not limitation, an expression cassette of the invention may comprise the cDNA expression cloning vectors, pCD and λNMT (Okayama H and Berg P (1983) Mol. Cell. Biol. 3: 280; Okayama H and Berg P (1985) Mol. Cell. Biol. 5: 1136, incorporated herein by reference). With reference to expression cassettes which are designed to function in chloroplasts, such as an expression cassette encoding a large subunit of Rubisco (rbcL) in a higher plant, the expression cassette comprises the sequences necessary to ensure expression in chloroplasts—typically the Rubisco L subunit encoding sequence is flanked by two regions of homology to the plastid genome so as to effect a homologous recombination with the chloroplastid genome; often a selectable marker gene is also present within the flanking plastid DNA sequences to facilitate selection of genetically stable transformed chloroplasts in the resultant transplastonic plant cells (see Maliga P (1993) TIBTECH 11: 101; Daniell et al. (1998) Nature Biotechnology 16:346, and references cited therein).

As used herein, the term “transcriptional unit” or “transcriptional complex” refers to a polynucleotide sequence that comprises a structural gene (exons), a cis-acting linked promoter and other cis-acting sequences necessary for efficient transcription of the structural sequences, distal regulatory elements necessary for appropriate tissue-specific and developmental transcription of the structural sequences, and additional cis sequences important for efficient transcription and translation (e.g., polyadenylation site, MRNA stability controlling sequences).

As used herein, the term “transcription regulatory region” refers to a DNA sequence comprising a functional promoter and any associated transcription elements (e.g., enhancer, CCAAT box, TATA box, LRE, ethanol-inducible element, etc.) that are essential for transcription of a polynucleotide sequence that is operably linked to the transcription regulatory region.

As used herein, the term “xenogeneic” is defined in relation to a recipient genome, host cell, or organism and means that an amino acid sequence or polynucleotide sequence is not encoded by or present in, respectively, the naturallyoccurring genome of the recipient genome, host cell, or organism. Xenogenic DNA sequences are foreign DNA sequences. Further, a nucleic acid sequence that has been substantially mutated (e.g., by site directed mutagenesis) is xenogeneic with respect to the genome from which the sequence was originally derived, if the mutated sequence does not naturally occur in the genome.

The term “corresponds to” is used herein to mean that a polynucleotide sequence is homologous (i.e., identical) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. For illustration, the nucleotide sequence “5′-TATAC” corresponds to a reference sequence “5′-TATAC” and is complementary to a reference sequence “5′-GTATA”.

The following terms are used to describe the sequence relationships between two or more polynucleotides: “reference sequence”, “comparison window”, “sequence identity”, “percentage of sequence identity”, and “substantial identity”. A “reference sequence” is a defined sequence used as a basis for a sequence comparison; a reference sequence may be a subset of a larger sequence, for example, as a segment of a full-length viral gene or virus genome. Generally, a reference sequence is at least 20 nucleotides in length, frequently at least 25 nucleotides in length, and often at least 50 nucleotides in length. Since two polynucleotides may each comprise (1) a sequence (i.e., a portion of the complete polynucleotide sequence) that is similar between the two polynucleotides, and (2) a sequence that is divergent between the two polynucleotides, sequence comparisons between two (or more) polynucleotides are typically performed by comparing sequences of the two polynucleotides over a “comparison window” to identify and compare local regions of sequence similarity.

A “comparison window”, as used herein, refers to a conceptual segment of at least 25 contiguous nucleotide positions wherein a polynucleotide sequence may be compared to a reference sequence of at least 25 contiguous nucleotides and wherein the portion of the polynucleotide sequence in the comparison window may comprise additions or deletions (i.e., gaps) of 20 percent or less as compared to the reference sequence (which for comparative purposes in this manner does not comprise additions or deletions) for optimal alignment of the two sequences. Optimal alignment of sequences for aligning a comparison window may be conducted by the local homology algorithm of Smith and Waterman (1981) Adv. Appl. Math. 2: 482, by the homology alignment algorithm of Needleman and Wunsch (1970) J. Mol. Biol. 48: 443, by the search for similarity method of Pearson and Lipman (1988)Proc. Natl. Acad. Sci. (U.S.A.) 85: 2444, by computerized implementations of these algorithms (GAP, BESTFIT, FASTA, and TFASTA in the Wisconsin Genetics Software Package Release 7.0, Genetics Computer Group, 575 Science Dr., Madison, Wis.), or by inspection, and the best alignment (i.e., resulting in the highest percentage of homology over the comparison window) generated by the various methods is selected.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or 1) occurs in both sequences to yield the number of matched positions, dividing the number of matched positions by the total number of positions in the window of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 89 to 95 percent sequence identity, more usually at least 99 percent sequence identity as compared to a reference sequence over a comparison window of at least 20 nucleotide positions, optionally over a window of at least 30-50 nucleotides, wherein the percentage of sequence identity is calculated by comparing the reference sequence to the polynucleotide sequence that may include deletions or additions which total 20 percent or less of the reference sequence over the window of comparison. The reference sequence may be a subset of a larger sequence.

Specific hybridization is defined herein as the formation, by hydrogen bonding or nucleotide (or nucleobase) bases, of hybrids between a probe polynucleotide (e.g., a polynucleotide of the invention and a specific target polynucleotide, wherein the probe preferentially hybridizes to the specific target such that, for example, a single band corresponding to, e.g., one or more of the RNA species of the gene (or specifically cleaved or processed RNA species) can be identified on a Northern blot of RNA prepared from a suitable source. Such hybrids may be completely or only partially base-paired. Polynucleotides of the invention which specifically hybridize to viral genome sequences may be prepared on the basis of the sequence data provided herein and available in the patent applications incorporated herein and scientific and patent publications noted above, and according to methods and thermodynamic principles known in the art and described in Sambrooke et al. et al., Molecular Cloning: A Laboratory Manual, 2nd Ed., (1989), Cold Spring Harbor, N.Y.; Berger and Kimmel, Methods in Enzymology, Volume 152, Guide to Molecular Cloning Techniques (1987), Academic Press, Inc., San Diego, Calif.; Goodspeed et al. (1989) Gene 76: 1; Dunn et al. (1989) J. Biol. Chem. 264: 13057, and Dunn et al. (1988) J. Biol. Chem. 263: 10878, which are each incorporated herein by reference.

“Physiological conditions” as used herein refers to temperature, pH, ionic strength, viscosity, and like biochemical parameters that are compatible with a viable plant organism or agricultural microorganism (e.g., Rhizobium, Agrobacterium, etc.), and/or that typically exist intracellularly in a viable cultured plant cell, particularly conditions existing in the nucleus of said cell. In general, in vitro physiological conditions can comprise 50-200 mM NaCl or KCI, pH 6.5-8.5, 20-45° C. and 0.001-10 mM divalent cation (e.g., Mg⁺⁺, Ca⁺⁺); preferably about 150 mM NaCl or KCI, pH 7.2-7.6, 5 mM divalent cation, and often include 0.01-1.0 percent nonspecific protein (e.g., BSA). A non-ionic detergent (Tween, NP-40, Triton X-100) can often be present, usually at about 0.001 to 2%, typically 0.05-0.2% (v/v). Particular aqueous conditions may be selected by the practitioner according to conventional methods. For general guidance, the following buffered aqueous conditions may be applicable: 10-250 mM NaCl, 5-50 mM Tris HCI, pH 5-8, with optional addition of divalent cation(s), metal chelators, nonionic detergents, membrane fractions, antifoam agents, and/or scintillants.

As used herein, the terms “label” or “labeled” refer to incorporation of a detectable marker, e.g., a radiolabeled amino acid or a recoverable label (e.g. biotinyl moieties that can be recovered by avidin or streptavidin). Recoverable labels can include covalently linked polynucleobase sequences that can be recovered by hybridization to a complementary sequence polynucleotide. Various methods of labeling polypeptides, PNAs, and polynucleotides are known in the art and may be used. Examples of labels include, but are not limited to, the following: radioisotopes (e.g., ³H, ¹⁴C, ³⁵S, ¹²⁵I, ¹³¹I), fluorescent or phosphorescent labels (e.g., FITC, rhodamine, lanthanide phosphors), enzymatic labels (e.g., horseradish peroxidase, β-galactosidase, luciferase, alkaline phosphatase), biotinyl groups, predetermined polypeptide epitopes recognized by a secondary reporter (e.g., leucine zipper pair sequences, binding sites for antibodies, transcriptional activator polypeptide, metal binding domains, epitope tags). In some embodiments, labels are attached by spacer arms of various lengths, e.g., to reduce potential steric hindrance.

As used herein, the term “statistically significant” means a result (i.e., an assay readout) that generally is at least two standard deviations above or below the mean of at least three separate determinations of a control assay readout and/or that is statistically significant as determined by Student's t-test or other art-accepted measure of statistical significance. To assess whether an enzyme has been significantly improved, e.g., has an activity that is significantly greater than a reference enzyme or a temperature stability that is significantly improved, one would look for a statistically significant difference in the properties of the enzyme as determined by an appropriate assay or measurement.

The term “transcriptional modulation” is used herein to refer to the capacity to either enhance transcription or inhibit transcription of a structural sequence linked in cis; such enhancement or inhibition may be contingent on the occurrence of a specific event, such as stimulation with an inducer and/or may only be manifest in certain cell types.

The term “agent” is used herein to denote a chemical compound, a mixture of chemical compounds, a biological macromolecule, or an extract made from biological materials such as bacteria, plants, fungi, or animal cells or tissues. Agents are evaluated for potential activity as Rubisco inhibitors or allosteric effectors by inclusion in screening assays described hereinbelow.

As used herein, “substantially pure” means an object species is the predominant species present (i.e., on a molar basis it is more abundant than any other individual macromolecular species in the composition), and preferably a substantially purified fraction is a composition wherein the object species comprises at least about 50 percent (on a molar basis) of all macromolecular species present. Generally, a substantially pure composition will comprise more than about 80 to 90 percent of all macromolecular species present in the composition. Most preferably, the object species is purified to essential homogeneity (contaminant species cannot be detected in the composition by conventional detection methods) wherein the composition consists essentially of a single macromolecular species. Solvent species, small molecules (<500 Daltons), and elemental ion species are not considered macromolecular species.

As used herein, the term “optimized” is used to mean substantially improved in a desired structure or function relative to an initial starting condition, not necessarily the optimal structure or function which could be obtained if all possible combinatorial variants could be made and evaluated, a condition which is typically impractical due to the number of possible combinations and permutations in polynucleotide sequences of significant length (e.g., a complete plant gene or genome).

As used herein, “Rubisco enzymatic phenotype” means an observable or otherwise detectable phenotype that can be discriminative based on Rubisco function. For example and not limitation, a Rubisco enzymatic phenotype can comprise an enzyme Km for a substrate, VO2, VCO2, V_{O2}/V_{CO2}, (V_{CO2}K_{O2}/V_{O2}K_{CO2}), K_{RuBP}, a turnover rate, an inhibition coefficient (Ki), or an observable or otherwise detectable trait that reports Rubisco function in a cell or clonal progeny thereof which otherwise lack said trait in the absence of significant Rubisco function.

As used herein, “Rubisco activase enzymatic phenotype” means an observable or otherwise detectable phenotype that can be discriminative based on Rubisco activase function. For example and not limitation, a Rubisco activase enzymatic phenotype can comprise an enzyme Km for a substrate, kcat, a turnover rate, an inhibition coefficient (Ki), thermal stability, temperature optimumm or an observable or otherwise detectable trait that reports Rubisco activase function in a cell or clonal progeny thereof which otherwise lack said trait in the absence of significant Rubisco activase function.

As used herein, “complementing subunit” is used principally with reference to Form I Rubisco composed of S and L subunits and means a Rubisco subunit of the opposite type (e.g., an S subunit can be a complementing subunit to an L subunit, and vice versa), wherein when the L and S subunits are present in a cell or in vitro reaction vessel under appropriate assay conditions they form a multimer having detectable Rubisco carboxylase activity. A complementing subunit can be obtained from the same taxonomic species of organism, or from a xenogenic species.

Calibration assays are performed to determine whether a selected first subunit is a complementing subunit with respect to a second subunit; if the first subunit produces a detectable allosteric effect upon the activity, it is deemed for purposes of this disclosure to constitute a complementing subunit.

### DESCRIPTION OF SPECIFIC EMBODIMENTS

The present invention provides methods, reagents, genetically modified plants, plant cells and protoplasts thereof, microbes, and polynucleotides, and compositions relating to the improvement of a photosynthetic carbon fixation enzyme, preferably a Rubisco, Rubisco variant or modified variant of one of these proteins.

In an aspect, the invention provides a shuffled Rubisco L subunit which is catalytically active in the presence of a complementing S subunit, which may itself be shuffled, and which exhibits an improved enzymatic profile, such as an increased Km for O₂, a decreased Km for CO₂, increased turnover rate for fixation of carbon, or the like. In an aspect, the shuffled L subunit is catalytically active in the absence of an S subunit and the presence of an S subunit does not significantly increase the catalytic activity of the L subunit as measured by RuBP carboxylase and/or RuBP oxygenase activity.

In another aspect, the invention provides a shuffled Rubisco activase, which may itself be shuffled, and which exhibits an improved enzymatic profile, such as an increased kcat, a decreased Km, elevated temperature optimun, or improved temperature stability. Improving the ability of Rubisco activase to activate Rubisco under high temperature could significantly increase the capacity of crop photosynthesis and maximize utilization of light at temperatures that are presently not considered to be optimal.

In a broad aspect, the invention is based, in part, on a method for shuffling polynucleotide sequences that encode a photosynthetic carbon fixation protein, such as Rubisco activase, a Rubisco subunit, such as a Form I rbcS subunit, a Form I rbcL subunit, or a Form II rbcL subunit, or combinations thereof. The method comprises the step of selecting at least one polynucleotide sequence that encodes a photosynthetic carbon fixation protein having an enhanced enzymatic phenotype and subjecting said selected polynucleotide sequence to at least one subsequent round of mutagenesis and/or sequence shuffling, and selection for the enhanced phenotype. Preferably, the method is performed recursively on a collection of selected polynucleotide sequences encoding the photosynthetic carbon fixation protein to iteratively provide polynucleotide sequences encoding photosynthetic carbon fixation protein species having the desired enhanced enzymatic phenotype.

The invention provides shuffled photosynthetic carbon fixation polynucleotides, wherein said shuffled sequences comprise at least 21 contiguous nucleotides, preferably at least 30 contiguous nucleotides, or more, of a first naturally occurring photosynthetic carbon fixation polynucleotide and at least 21 contiguous nucleotides, preferably at least 30 contiguous nucleotides, or more, of a second naturally occurring photosynthetic carbon fixation polynucleotide, operably linked in reading frame to encode a photosynthetic carbon fixation protein which has an enhanced enzymatic phenotype. In some variations, it will be possible to use shuffled encoding sequences which have less than 21 contiguous nucleotides identical to a naturally-occurring gene sequence.

The invention provides shuffled rbcS encoding sequences, wherein the shuffled sequences comprise portions of a first parental rbcS encoding sequence which comprises at least one mutation in the encoding sequence as compared to the 10 collection of predetermined naturally occurring rbcS sequences.

Generally, the nomenclature used hereafter and the laboratory procedures in cell culture, molecular genetics, virology, and nucleic acid chemistry and hybridization described below are those well known and commonly employed in the art. Standard techniques are used for recombinant nucleic acid methods, 15 polynucleotide synthesis, and microbial culture and transformation (e.g., biolistics, Agrobacterium (Ti plasmid), electroporation, lipofection). Generally enzymatic reactions and purification steps are performed according to the manufacturer's specifications. The techniques and procedures are generally performed according to conventional methods in the art and various general references (see, generally, Sambrook et al. Molecular Cloning: A Laboratory Manual, 2d ed. (1989) Cold Spring Harbor Laboratory Press, Cold Spring Harbor, N.Y., which is incorporated herein by reference) which are provided throughout this document. The procedures therein are believed to be well known in the art and are provided for the convenience of the reader. All the information contained therein is incorporated herein by reference. Oligonucleotides can be synthesized on an Applied Bio Systems oligonucleotide synthesizer according to specifications provided by the manufacturer.

Methods for PCR amplification are described in the art (PCR Technology: Principles and Applications for DNA Amplification ed. HA Erlich, Freeman Press, New York, NY (1992); PCR Protocols: A Guide to Methods and Applications, eds. Innis, Gelfland, Snisky, and White, Academic Press, San Diego, Calif. (1990); Mattila et al. (1991) Nucleic Acids Res. 19: 4967; Eckert, K.A. and Kunkel, T. A. (1991) PCR Methods and Applications 1: 17; PCR, eds. McPherson, Quirkes, and Taylor, IRL Press, Oxford; and U.S. Pat. No. 4,683,202, which are incorporated herein by reference). Leaf PCR is suitable for genotype analysis of transgenote plants.

All sequences referred to herein or equivalents which function in the disclosed methods can be retrieved by GenBank database file designation or a commonly used reference name which is indexed in GenBank or otherwise published are incorporated herein by reference and are publicly available. Over 1,000 Rubisco homologues are available, e.g., in GenBank. Many Rubisco activase genes (rca) from 10 various species including Arabidopsis, rice, wheat, spinach, Phaseolus vugaris and tobacco have been sequenced and deposited in GenBank. The genes can be obtained by either RT-PCR, PCR from a cDNA library, synthesis, or other methods known in the art.

### Incorporation by Reference of Related Applications

The following patents, patent applications and publications are incorporated herein by reference for all purposes:

Soong, N. et al. (2000) “Molecular breeding of viruses” Nat Genet 25(4):436-39; Stemmer, et al. (1999) “Molecular breeding of viruses for targeting and other clinical properties” Tumor Targeting 4:1-4; Ness et al. (1999) “DNA Shuffling of subgenomic sequences of subtilisin” Nature Biotechnology 17:893-896; Chang et al. (1999) “Evolution of a cytokine using DNA family shuffling” Nature Biotechnology 17:793-797; Minshull and Stemmer (1999) “Protein evolution by molecular breeding” Current Opinion in Chemical Biology 3:284-290; Christians et al. (1999) “Directed evolution of thymidine kinase for AZT phosphorylation using DNA family shuffling” Nature Biotechnology 17:259-264; Crameri et al. (1998) “DNA shuffling of a family of genes from diverse species accelerates directed evolution” Nature 391:288-291; Crameri et al. (1997) “Molecular evolution of an arsenate detoxification pathway by DNA shuffling,” Nature Biotechnology 15:436-438; Zhang et al. (1997) “Directed evolution of an effective fucosidase from a galactosidase by DNA shuffling and screening” Proc. Natl. Acad. Sci. USA 94:4504-4509; Patten et al. (1997) “Applications of DNA Shuffling to Pharmaceuticals and Vaccines” Current Opinion in Biotechnology 8:724-733; Crameri et al. (1996) “Construction and evolution of antibody-phage libraries by DNA shuffling” Nature Medicine 2:100-103; Crameri et al. (1996) “Improved green fluorescent protein by molecular evolution using DNA shuffling” Nature Biotechnology 14:315-319; Gates et al. (1996) “Affinity selective isolation of ligands from peptide libraries through display on a lac repressor ‘headpiece dimer’” Journal of Molecular Biology 255:373-386; Stemmer (1996) “Sexual PCR and Assembly PCR” In: The Encyclopedia of Molecular Biology. VCH Publishers, New York. pp.447-457; Crameri and Stemmer (1995) “Combinatorial multiple cassette mutagenesis creates all the permutations of mutant and wildtype cassettes” BioTechniques 18:194-195; Stemmer et al., (1995) “Single-step assembly of a gene and entire plasmid form large numbers of oligodeoxy-ribonucleotides” Gene, 164:49-53; Stemmer (1995) “The Evolution of Molecular Computation” Science 270: 1510; Stemmer (1995) “Searching Sequence Space” Bio/Technology 13:549-553; Stemmer (1994) “Rapid evolution of a protein in vitro by DNA shuffling” Nature 370:389-391; and Stemmer (1994) “DNA shuffling by random fragmentation and reassembly: In vitro recombination for molecular evolution.” Proc. Natl. Acad. Sci. USA 91:10747-10751; (Ling et al. (1997) “Approaches to DNA mutagenesis: an overview” Anal Biochem. 254(2): 157-178; Dale et al. (1996) “Oligonucleotide-directed random mutagenesis using the phosphorothioate method” Methods Mol. Biol. 57:369-374; Smith (1985) “In vitro mutagenesis” Ann. Rev. Genet. 19:423-462; Botstein & Shortle (1985) “Strategies and applications of in vitro mutagenesis” Science 229:1193-1201; Carter (1986) “Site-directed mutagenesis” Biochem. J. 237:1-7; and Kunkel (1987) “The efficiency of oligonucleotide directed mutagenesis” in Nucleic Acids & Molecular Biology (Eckstein, F. and Lilley, D.M.J. eds., Springer Verlag, Berlin)); mutagenesis using uracil containing templates (Kunkel (1985) “Rapid and efficient site-specific mutagenesis without phenotypic selection” Proc. Natl. Acad. Sci. USA 82:488-492; Kunkel et al. (1987) “Rapid and efficient site-specific mutagenesis without phenotypic selection” Methods in Enzymol. 154, 367-382; and Bass et al. (1988) “Mutant Trp repressors with new DNA-binding specificities” Science 242:240-245); oligonucleotide-directed mutagenesis (Methods in Enzymol. 100: 468-500 (1983); Methods in Enzymol. 154: 329-350 (1987); Zoller & Smith (1982) “Oligonucleotide-directed mutagenesis using M13-derived vectors:

an efficient and general procedure for the production of point mutations in any DNA fragment” Nucleic Acids Res. 10:6487-6500; Zoller & Smith (1983) “Oligonucleotide-directed mutagenesis of DNA fragments cloned into M13 vectors” Methods in Enzymol. 100:468-500; and Zoller & Smith (1987) “Oligonucleotide-directed mutagenesis: a simple method using two oligonucleotide primers and a single-stranded DNA template” Methods in Enzymol. 154:329-350); phosphorothioate-modified DNA mutagenesis (Taylor et al. (1985) “The use of phosphorothioate-modified DNA in restriction enzyme reactions to prepare nicked DNA” Nucl. Acids Res. 13: 8749-8764; Taylor et al. (1985) “The rapid generation of oligonucleotide-directed mutations at high frequency using phosphorothioate-modified DNA” Nucl. Acids Res. 13: 8765-8787 (1985); Nakamaye & Eckstein (1986) “Inhibition of restriction endonuclease Nci I cleavage by phosphorothioate groups and its application to oligonucleotide-directed mutagenesis” Nucl. Acids Res. 14: 9679-9698; Sayers et al. (1988) “Y-T Exonucleases in phosphorothioate-based oligonucleotide-directed mutagenesis” Nucl. Acids Res. 16:791-802; and Sayers et al. (1988) “Strand specific cleavage of phosphorothioate-containing DNA by reaction with restriction endonucleases in the presence of ethidium bromide” Nucl. Acids Res. 16: 803-814); mutagenesis using gapped duplex DNA (Kramer et al. (1984) “The gapped duplex DNA approach to oligonucleotide-directed mutation construction” Nucl. Acids Res. 12: 9441-9456; Kramer & Fritz (1987) Methods in Enzymol. “Oligonucleotide-directed construction of mutations via gapped duplex DNA” 154:350-367; Kramer et al. (1988) “Improved enzymatic in vitro reactions in the gapped duplex DNA approach to oligonucleotide-directed construction of mutations” Nucl. Acids Res. 16: 7207; and Fritz et al. (1988) “Oligonucleotide-directed construction of mutations: a gapped duplex DNA procedure without enzymatic reactions in vitro” Nucl. Acids Res. 16: 6987-6999); Kramer et al. (1984) “Point Mismatch Repair” Cell 38:879-887), mutagenesis using repair-deficient host strains (Carter et al. (1985) “Improved oligonucleotide site-directed mutagenesis using M13 vectors” Nucl. Acids Res. 13: 4431-4443; and Carter (1987) “Improved oligonucleotide-directed mutagenesis using M13 vectors” Methods in Enzymol. 154: 382-403), deletion mutagenesis (Eghtedarzadeh & Henikoff (1986) “Use of oligonucleotides to generate large deletions” Nucl. Acids Res. 14: 5115), restrictionselection and restriction-selection and restriction-purification (Wells et al. (1986) “Importance of hydrogen-bond formation in stabilizing the transition state of subtilisin” Phil. Trans. R. Soc. Lond. A 317: 415-423), mutagenesis by total gene synthesis (Nambiar et al. (1984) “Total synthesis and cloning of a gene coding for the ribonuclease S protein” Science 223: 1299-1301; Sakamar and Khorana (1988) “Total synthesis and expression of a gene for the a-subunit of bovine rod outer segment guanine nucleotide-binding protein (transducin)” Nucl. Acids Res. 14: 6361-6372; Wells et al. (1985) “Cassette mutagenesis: an efficient method for generation of multiple mutations at defined sites” Gene 34:315-323; and Grundstrom et al. (1985) “Oligonucleotide-directed mutagenesis by microscale ‘shot-gun’ gene synthesis” Nucl. Acids Res. 13: 3305-3316), double-strand break repair (Mandecki (1986); Arnold (1993) “Protein engineering for unusual environments” Current Opinion in Biotechnology 4:450-455. “Oligonucleotide-directed double-strand break repair in plasmids of Escherichia coli: a method for site-specific mutagenesis” Proc. Natl. Acad. Sci. USA, 83:7177-7181; U.S. Pat. No. 5,605,793 to Stemmer (Feb. 25, 1997), “Methods for In Vitro Recombination;” U.S. Pat. No. 5,811,238 to Stemmer et al. (Sep. 22, 1998) “Methods for Generating Polynucleotides having Desired Characteristics by Iterative Selection and Recombination;” U.S. Pat. No. 5,830,721 to Stemmer et al. (Nov. 3, 1998), “DNA Mutagenesis by Random Fragmentation and Reassembly;” U.S. Pat. No. 5,834,252 to Stemmer, et al. (Nov. 10, 1998) “End-Complementary Polymerase Reaction;” U.S. Pat. No. 5,837,458 to Minshull, et al. (Nov. 17, 1998), “Methods and Compositions for Cellular and Metabolic Engineering;” WO 95/22625, Stemmer and Crameri, “Mutagenesis by Random Fragmentation and Reassembly;” WO 96/33207 by Stemmer and Lipschutz “End Complementary Polymerase Chain Reaction;” WO 97/20078 by Stemmer and Crameri “Methods for Generating Polynucleotides having Desired Characteristics by Iterative Selection and Recombination;” WO 97/35966 by Minshull and Stemmer, “Methods and Compositions for Cellular and Metabolic Engineering;” WO 99/41402 by Punnonen et al. “Targeting of Genetic Vaccine Vectors;” WO 99/41383 by Punnonen et al. “Antigen Library Immunization;” WO 99/41369 by Punnonen et al. “Genetic Vaccine Vector Engineering;” WO 99/41368 by Punnonen et al. “Optimization of Immunomodulatory Properties of Genetic Vaccines;” EP 752008 by Stemmer and Crameri, “DNA Mutagenesis by Random Fragmentation and Reassembly;” EP 0932670 by Stemmer “Evolving Cellular DNA Uptake by Recursive Sequence Recombination;” WO 99/23107 by Stemmer et al., “Modification of Virus Tropism and Host Range by Viral Genome Shuffling;” WO 99/21979 by Apt et al., “Human Papillomavirus Vectors;” WO 98/31837 by del Cardayre et al. “Evolution of Whole Cells and Organisms by Recursive Sequence Recombination;” WO 98/27230 by Patten and Stemmer, “Methods and Compositions for Polypeptide Engineering;” WO 98/13487 by Stemmer et al., “Methods for Optimization of Gene Therapy by Recursive Sequence Shuffling and Selection,” WO 00/00632, “Methods for Generating Highly Diverse Libraries,” WO 00/09679, “Methods for Obtaining in Vitro Recombined Polynucleotide Sequence Banks and Resulting Sequences,” WO 98/42832 by Arnold et al., “Recombination of Polynucleotide Sequences Using Random or Defined Primers,” WO 99/29902 by Arnold et al., “Method for Creating Polynucleotide and Polypeptide Sequences,” WO 98/41653 by Vind, “An in Vitro Method for Construction of a DNA Library,” WO 98/41622 by Borchert et al., “Method for Constructing a Library Using DNA Shuffling,” and WO 98/42727 by Pati and Zarling, “Sequence Alterations using Homologous Recombination,” WO 00/18906 by Patten et al., “Shuffling of Codon-Altered Genes;” WO 00/04190 by del Cardayre et al. “Evolution of Whole Cells and Organisms by Recursive Recombination;” WO 00/42561 by Crameri et al., “Oligonucleotide Mediated Nucleic Acid Recombination;” WO 00/42559 by Selifonov and Stemmer “Methods of Populating Data Structures for Use in Evolutionary Simulations;” WO 00/42560 by Selifonov et al., “Methods for Making Character Strings, Polynucleotides & Polypeptides Having Desired Characteristics;” WO 01/23401 by Welch et al., “Use of Codon-Varied Oligonucleotide Synthesis for Synthetic Shuffling;” and PCT/US01/06775 “Single-Stranded Nucleic Acid Template-Mediated Recombination and Nucleic Acid Fragment Isolation” by Afflholter; SHUFFLING OF CODON ALTERED GENES” by Patten et al. filed Sep. 28, 1999, (U.S. Ser. No. 09/407,800); “EVOLUTION OF WHOLE CELLS AND ORGANISMS BY RECURSIVE SEQUENCE RECOMBINATION”, by del Cardayre et al. filed Jul. 15, 1998 (U.S. Ser. No. 09/166,188), and Jul. 15, 1999 (U.S. Ser. No. 09/354,922); “OLIGONUCLEOTIDE MEDIATED NUCLEIC ACID RECOMBINATION” by Crameri et al., filed Sep. 28, 1999 (U.S. Ser. No. 09/408,392), and “OLIGONUCLEOTIDE MEDIATED NUCLEIC ACID RECOMBINATION” by Crameri et al., filed Jan. 18, 2000 (PCT/US00/01203); “USE OF CODON-BASED OLIGONUCLEOTIDE SYNTHESIS FOR SYNTHETIC SHUFFLING” by Welch et al., filed Sep. 28, 1999 (U.S. Ser. No. 09/408,393); “METHODS FOR MAKING CHARACTER STRINGS, POLYNUCLEOTIDES & POLYPEPTIDES HAVING DESIRED CHARACTERISTICS” by Selifonov et al., filed Jan. 18, 2000, (PCT/US00/01202) and, e.g., “METHODS FOR MAKING CHARACTER STRINGS, POLYNUCLEOTIDES & POLYPEPTIDES HAVING DESIRED CHARACTERISTICS” by Selifonov et al., filed Jul. 18, 2000 (U.S. Ser. No. 09/618,579); “METHODS OF POPULATING DATA STRUCTURES FOR USE IN EVOLUTIONARY SIMULATIONS” by Selifonov and Stemmer (PCT/US00/01138), filed Jan. 18, 2000; and “SINGLE-STRANDED NUCLEIC ACID TEMPLATE-MEDIATED RECOMBINATION AND NUCLEIC ACID FRAGMENT ISOLATION” by Affholter (U.S. Ser. No. 60/186,482, filed Mar. 2, 2000); WO 98/31837 by del Cardayre et al. “Evolution of Whole Cells and Organisms by Recursive Sequence Recombination;” and in, e.g., PCT/US99/15972 by del Cardayre et al., also entitled “Evolution of Whole Cells and Organisms by Recursive Sequence Recombination; WO 00/42561 by Crameri et al., “Olgonucleotide Mediated Nucleic Acid Recombination;” WO 01/23401 by Welch et al., “Use of Codon-Varied Oligonucleotide Synthesis for Synthetic Shuffling;” WO 00/42560 by Selifonov et al., “Methods for Making Character Strings, Polynucleotides and Polypeptides Having Desired Characteristics;” and WO 00/42559 by Selifonov and Stemmer “Methods of Populating Data Structures for Use in Evolutionary Simulations; WO 00/42560 by Selifonov et al., “Methods for Making Character Strings, Polynucleotides and Polypeptides Having Desired Characteristics” and WO 00/42559 by Selifonov and Stemmer “Methods of Populating Data Structures for Use in Evolutionary Simulations; PCT/US01/06775; U.S. Pat. No. 5,965,408; Ostermeier et al. (1999) “A combinatorial approach to hybrid enzymes independent of DNA homology” Nature Biotech 17:1205; Ostermeier et al. (1999) “Combinatorial Protein Engineering by Incremental Truncation,” Proc. Natl. Acad. Sci. USA, 96: 3562-67; Ostermeier et al. (1999), “Incremental Truncation as a Strategy in the Engineering of Novel Biocatalysts,” Biological and Medicinal Chemistry, 7: 2139-44; Leung et al. (1989) Technique 1:11-15 and Caldwell et al. (1992) PCR Methods Applic. 2:28-33; Reidhaar-Olson et al. (1988) Science, 241:53-57; Delegrave & Youvan (1993) Biotechnology Research 11: 1548-1552; U.S. Pat. No. 5,756,316; Peterson et al. (1998) U.S. Pat. No. 5,783,431 “METHODS FOR GENERATING AND SCREENING NOVEL METABOLIC PATHWAYS,” and Thompson, et al. (1998) U.S. Pat. No. 5,824,485 METHODS FOR GENERATING AND SCREENING NOVEL METABOLIC PATHWAYS),Short (1999) U.S. Pat. No. 5,958,672 “PROTEIN ACTIVITY SCREENING OF CLONES HAVING DNA FROM UNCULTIVATED MICROORGANISMS”; U.S. Pat. No. 5,939,250; PCT applications WO 99/10539 and WO 00/46344; Arkin and Youvan (1992) “Optimizing nucleotide mixtures to encode specific subsets of amino acids for semi-random mutagenesis” Biotechnology 10:297-300; Reidhaar-Olson et al. (1991) “Random mutagenesis of protein sequences using oligonucleotide cassettes” Methods Enzymol. 208:564-86; Lim and Sauer (1991) “The role of internal packing interactions in determining the structure and stability of a protein” J. Mol. Biol. 219:359-76; Breyer and Sauer (1989) “Mutational analysis of the fine specificity of binding of monoclonal antibody 51F to lambda repressor” J. Biol. Chem. 264:13355-60); and “Walk-Through Mutagenesis” (Crea, R; U.S. Pat. Nos. 5,830,650 and 5,798,208, and EP Patent 0527809 B1.

### Overview

The invention relates in part to a method for generating novel or improved photosynthetic carbon fixation proteins and photosynthetic carbon fixation polynucleotides, and improved carbon fixation phenotypes which do not naturally occur or would not be expected to occur at a substantial frequency in nature. A broad aspect of the method employs recursive nucleotide sequence recombination, termed “sequence shuffling” which enables the rapid generation of a collection of broadly diverse phenotypes that can be selectively bred for a broader range of novel phenotypes or more extreme phenotypes than would otherwise occur by natural evolution in the same time period. A basic variation of the method is a recursive process comprising: (1) sequence shuffling of a plurality of species of a genetic sequence, which species may differ by as little as a single nucleotide difference or may be substantially different yet retain sufficient regions of sequence similarity or site-specific recombination junction sites to support shuffling recombination, (2) selection of the resultant shuffled genetic sequence to isolate or enrich a plurality of shuffled genetic sequences having a desired phenotype(s), and (3) repeating steps (1) and (2) on the plurality of shuffled genetic sequences having the desired phenotype(s) until one or more variant genetic sequences encoding a sufficiently optimized desired phenotype is obtained. In this general manner, the method facilitates the “forced evolution” of a novel or improved genetic sequence to encode a desired Rubisco or Rubisco activase enzymatic phenotype which natural selection and evolution has heretofore not generated in the reference agricultural organism.

Typically, a plurality of photosynthetic carbon fixation polynucleotides are shuffled and selected by the present method. The method can be used with a plurality of alleles, homologs, or cognate genes of a genetic locus, or even with a plurality or genetic sequences from related organisms, and in some instances with unrelated genetic sequences or portions thereof which have recombinogenic portions (either naturally or generated via genetic engineering). Furthermore, the method can be used to evolve a heterologous Rubisco or Rubisco activase sequence (e.g., a non-naturally occurring mutant gene, or a subunit from another species) to optimize its function in concert with a complementing subunit, and/or in a particular host cell.

Shufflants of photosynthetic carbon fixation polynucleotides are generated by any suitable shuffling method as noted above from one or more parental sequences, optionally including mutagenesis, in vitro manipulation, in vivo manipulation of sequences or in silico manipulation of sequences, and the resultant shufflants are introduced into a suitable host cell, typically in the form of expression cassettes wherein the shuffled polynucleotide sequence encoding the polypeptide is operably linked to a transcriptional regulatory sequence and any necessary sequences for ensuring transcription, translation, and processing of the encoded polypeptide. Each such expression cassette or its shuffled photosynthetic carbon fixation polynucleotide can be referred to as a “library member” composing a library of shuffled Rubisco or Rubisco activase sequences. The library is introduced into a population of host cells, such that individual host cells receive substantially one or a few species of library member(s), to form a population of shufflant host cells expressing a library of shuffled photosynthetic carbon fixation protein species. The population of shufflant host cells is screened so as to isolate or segregate host cells and/or their progeny which express enzymes having the desired enhanced phenotype. The shuffled sequence(s) is/are recovered from the isolated or segregated shufflant host cells, and typically subjected to at least one subsequent round of mutagenesis and/or sequence shuffling, introduced into suitable host cells, and selected for the desired enhanced enzymatic phenotype; this cycle is generally performed iteratively until the shufflant host cells express a photosynthetic carbon fixation protein having the desired level or enzymatic phenotype or until the rate of improvement in the desired enzymatic phenotype produced by shuffling has substantially plateaued. The shufflant polynucleotides expressed in the host cells following the iterative process of shuffling and selection encode polypeptide specie(s) having the desired enhanced phenotype.

For illustration and not to limit the invention, examples of a desired Rubisco enzymatic phenotype can include increased RuBP carboxylase rate, decreased RuBP oxygenase rate, increased Km for O₂, decreased Km for CO₂, decreased ratio of Km for CO₂ to Km for O₂, velocity for O₂ or CO₂, and the like as described herein and as may be desired by the skilled artisan.

A variety of Rubisco gene and gene homologue sources are known and can be used in the recombination processes herein. For example, as noted, a variety of references herein describe such genes. For example, Croy, (ed.) (1993) Plant Molecular Biology Bios Scientific Publishers, Oxford, U.K. describe several Rubisco genes and sequence sources in public databases. Examples of public databases that include Rubisco sources include: Genbank: www.ncbi.nlm.nih.gov/genbank/; EMBL: www.ebi.ac.uk.embl/; as well as, e.g., the protein databank, Brookhaven Laboratories; the University of Wisconsin Biothechology Center, the DNA databank of Japan, Laboratory of genetic Information Research, Misuina, Shizuda, Japan. As noted, over 1,000 different Rubisco homologues are available in Genbank alone. In addition, specific internet sites which provide information regarding Rubisco include, e.g., http://ss.tnaes.affrc.go.jp/pub/suzuki/Rubisco.html; http://icdweb.cc.purdue.edu/˜knollje/Rubisco.html; http://www.agron.missouri.edu/cgi-bin/sybgw_mdb/mdb3/Locus/114858; http://gdb.wehi.edu.au/scop/data/scop.1.004.037.001.000.000.html; http://www.blc.arizona.edu/courses/181gh/rick/photosynthesis/Calvin.html; http://www.tarweed.com/pgr/PGR98-207.html; and http://homepage.ruhr-uni-bochum.de/Marc.Saric/Rubisco3.html.

Many Rubisco activase genes (rca) from various species including Arabidopsis, rice, wheat, spinach, Phaseolus vugaris and tobacco have been sequenced and deposited in GenBank. The genes can be obtained by either RT-PCR, PCR from a cDNA library, synthesis, or other methods known in the art.

The following publications describe a variety of recursive recombination procedures and/or methods which can be incorporated into such procedures, e.g., for shuffling of Rubisco or Rubisco activase genes and gene fragments as herein: Stemmer, et al., (1999) Molecular breeding of viruses for targeting and other clinical properties. Tumor Targeting 4:1-4; Nesset al. (1999) DNA Shuffling of subgenomic sequences of subtilisin Nature Biotechnology 17:893-896; Chang et al. (1999) Evolution of a cytokine using DNA family shuffling Nature Biotechnology 17:793-797; Minshull and Stemmer (1999) Protein evolution by molecular breeding Current Opinion in Chemical Biology 3:284-290; Christians et al. (1999) Directed evolution of thymidine kinase for AZT phosphorylation using DNA family shuffling Nature Biotechnology 17:259-264; Crameriet al. (1998) ADNA shuffling of a family of genes from diverse species accelerates directed evolution Nature 391:288-291;-Crameri et al. (1997) Molecular evolution of an arsenate detoxification pathway by DNA shuffling, Nature Biotechnology 15:436-438; Zhang et al. (1997) Directed evolution of an effective fucosidase from a galactosidase by DNA shuffling and screening Proceedings of the National Academy of Sciences, U.S.A. 94:4504-4509; Patten et al. (1997) Applications of DNA Shuffling to Pharmaceuticals and Vaccines Current Opinion in Biotechnology 8:724-733; Crameri et al. (1996) Construction and evolution of antibody-phage libraries by DNA shuffling Nature Medicine 2:100-103; Crameri et al. (1996) Improved green fluorescent protein by molecular evolution using DNA shuffling Nature Biotechnology 14:315-319; Gates et al. (1996) Affinity selective isolation of ligands from peptide libraries through display on a lac repressor ‘headpiece dimer’ Journal of Molecular Biology 255:373-386; Stemmer (1996) Sexual PCR and Assembly PCR In: The Encyclopedia of Molecular Biology. VCH Publishers, New York. pp.447-457; Crameri and Stemmer (1995) Combinatorial multiple cassette mutagenesis creates all the permutations of mutant and wildtype cassettes BioTechniques 18:194-195; Stemmer et al., (1995) Single-step assembly of a gene and entire plasmid form large numbers of oligodeoxyribonucleotides Gene, 164:49-53; Stemmer (1995) The Evolution of Molecular Computation Science 270: 1510; Stemmer (1995) Searching Sequence Space Bio/Technology 13:549-553; Stemmer (1994) Rapid evolution of a protein in vitro by DNA shuffling Nature 370:389-391; and Stemmer (1994) DNA shuffling by random fragmentation and reassembly: In vitro recombination for molecular evolution. Proceedings of the National Academy of Sciences, U.S.A. 91:10747-10751.

Additional details regarding DNA shuffling methods are found in U.S. Patents by the inventors and their co-workers, including: U.S. Pat. No. 5,605,793 to Stemmer (Feb. 25, 1997), METHODS FOR IN VITRO RECOMBINATION; U.S. Pat. No. 5,811,238 to Stemmer et al. (Sep. 22, 1998) METHODS FOR GENERATING POLYNUCLEOTIDES HAVING DESIRED CHARACTERISTICS BY ITERATIVE SELECTION AND RECOMBINATION; U.S. Pat. No. 5,830,721 to Stemmer et al. (Nov. 3, 1998), DNA MUTAGENESIS BY RANDOM FRAGMENTATION AND REASSEMBLY; U.S. Pat. No. 5,834,252 to Stemmer, et al. (Nov. 10, 1998) END-COMPLEMENTARY POLYMERASE REACTION, and U.S. Pat. No. 5,837,458 to Minshull, et al. (Nov. 17, 1998), METHODS AND COMPOSITIONS FOR CELLULAR AND METABOLIC ENGINEERING.

In addition, details and formats for DNA shuffling are found in a variety of PCT and foreign patent application publications, including: Stemmer and Crameri, DNA MUTAGENESIS BY RANDOM FRAGMENTATION AND REASEMBLY WO 95/22625; Stemmer and Lipschutz END COMPLEMENTARY POLYMERASE CHAIN REACTION WO 96/33207; Stemmer and Crameri METHODS FOR GENERATING POLYNUCLEOTIDES HAVING DESIRED CHARACTERISTICS BY ITERATIVE SELECTION AND RECOMBINATION WO 97/0078; Minshul and Stemmer, METHODS AND COMPOSITIONS FOR CELLULAR AND METABOLIC ENGINEERING WO 97/35966; Punnonen et al. TARGETING OF GENETIC VACCINE VECTORS WO 99/41402; Punnonen et al. ANTIGEN LIBRARY IMMUNIZATION WO 99/41383; Punnonen et al. GENETIC VACCINE VECTOR ENGINEERING WO 99/41369; Punnonen et al. OPTIMIZATION OF IMMUNOMODULATORY PROPERTIES OF GENETIC VACCINES WO 9941368; Stemmer and Crameri, DNA MUTAGENESIS BY RANDOM FRAGMENTATION AND REASSEMBLY EP 0934999; Stemmer EVOLVING CELLULAR DNA UPTAKE BY RECURSIVE SEQUENCE RECOMBINATION EP 0932670; Stemmer et al., MODIFICATION OF VIRUS TROPISM AND HOST RANGE BY VIRAL GENOME SHUFFLING WO 9923107; Apt et al., HUMAN PAPILLOMAVIRUS VECTORS WO 9921979; Del Cardayre et al. EVOLUTION OF WHOLE CELLS AND ORGANISMS BY RECURSIVE SEQUENCE RECOMBINATION WO 9831837; Patten and Stemmer, METHODS AND COMPOSITIONS FOR POLYPEPTIDE ENGINEERING WO 9827230; Stemmer et al., and METHODS FOR OPTIMIZATION OF GENE THERAPY BY RECURSIVE SEQUENCE SHUFFLING AND SELECTION WO 9813487.

Certain U.S. applications provide additional details regarding DNA shuffling and related techniques, including SHUFFLING OF CODON ALTERED GENES by Patten et al. filed Sep. 29, 1998, (U.S. Ser. No. 60/102,362), Jan. 29, 30, 1999 (U.S. Ser. No. 60/117,729), and Sep. 28, 1999, U.S. Ser. No. 09/407,800 (Attorney Docket Number 20-28520US/PCT); EVOLUTION OF WHOLE CELLS AND ORGANISMS BY RECURSIVE SEQUENCE RECOMBINATION, by del Cardyre et al. filed Jul. 15, 1998 (U.S. Ser. No. 09/166,188), and Jul. 15, 1999 (U.S. Ser. No. 09/354,922); OLIGONUCLEOTIDE MEDIATED NUCLEIC ACID RECOMBINATION by Crameri et al., filed Feb. 5, 1999 (U.S. Ser. No. 60/118,813) and filed Jun. 24, 1999 (U.S. Ser. No. 60/141,049) and filed Sep. 28, 1999 (U.S. Ser. No. 09/408,392, Attorney Docket Number 02-29620US); and USE OF CODON-BASED OLIGONUCLEOTIDE SYNTHESIS FOR SYNTHETIC SHUFFLING by Welch et al., filed Sep. 28, 1999 (U.S. Ser. No. 09/408,393, Attorney Docket Number 02-010070US); and METHODS FOR MAKING CHARACTER STRINGS, POLYNUCLEOTIDES & POLYPEPTIDES HAVING DESIRED CHARACTERISTICS by Selifonov and Stemmer, filed Feb. 5, 1999 (U.S. Ser. No. 60/118854) and METHODS FOR MAKING CHARACTER STRINGS, POLYNUCLEOTIDES & POLYPEPTIDES HAVING DESIRED CHARACTERISTICS by Selifonov et al. filed Oct. 12, 1999 (U.S. Ser. No. 09/416375). As review of the foregoing publications, patents, published applications and U.S. patent applications reveals, recursive recombination and selection of nucleic acids to provide new nucleic acids with desired properties can be carried out by a number of established methods. Any of these methods can be adapted to the present invention to evolve photosynthetic carbon fixation polynucleotides or homolgues to produce new enzymes with improved properties. Both the methods of making such enzymes and the enzymes or enzyme coding libraries produced by these methods are a feature of the invention.

In brief, at least 5 different general classes of recombination methods are applicable to the present invention. First, nucleic acids can be recombined in vitro by any of a variety of techniques discussed in the references above, including e.g., DNAse digestion of nucleic acids to be recombined followed by ligation and/or PCR reassembly of the nucleic acids. Second, nucleic acids can be recursively recombined in vivo, e.g., by allowing recombination to occur between nucleic acids in cells. Third, whole cell genome recombination methods can be used in which whole genomes of cells are recombined, optionally including spiking of the genomic or chloroplast recombination mixtures with desired library components such as Rubisco encoding nucleic acids. Fourth, synthetic recombination methods can be used, in which oligonucleotides corresponding to different photosynthetic carbon fixation polynucleotide homologues are synthesized and reassembled in PCR or ligation reactions which include oligonucleotides which correspond to more than one parental nucleic acid, thereby generating new recombined nucleic acids. Oligonucleotides can be made by standard nucleotide addition methods, or can be made, e.g., by trinucleotide synthetic approaches. Fifth, in silico methods of recombination can be effected in which genetic algorithms are used in a computer to recombine sequence strings which correspond to photosynthetic carbon fixation protein homologues. The resulting recombined sequence strings are optionally converted into nucleic acids by synthesis of nucleic acids which correspond to the recombined sequences, e.g., in concert with oligonucleotide synthesis/ gene reassembly techniques. Any of the preceding general recombination formats can be practiced in a reiterative fashion to generate a more diverse set of recombinant nucleic acids.

The above references provide these and other basic recombination formats as well as many modifications of these formats. Regardless of the format which is used, the nucleic acids of the invention can be recombined (with each other or with related (or even unrelated) nucleic acids to produce a diverse set of recombinant nucleic acids, including homologous nucleic acids.

Following recombination, any nucleic acids which are produced can be selected for a desired activity. A variety of related (or even unrelated) properties can be assayed for, using any available assay.

One basic format of shuffling consists of a method for generating a selected polynucleotide sequence or population of selected polynucleotide sequences, typically in the form of amplified and/or cloned polynucleotides, whereby the selected polynucleotide sequence(s) possess or encode a desired phenotypic characteristic (e.g., encode a polypeptide, promote transcription of linked polynucleotides, modify transformation efficiency, bind a protein, and the like) which can be selected for. One method of identifying polypeptides that possess a desired structural or functional property, such as encoding a desired enzymatic function(s) (e.g., an enhanced Rubisco, a herbicide catabolizing enzyme, an optimized plant biosynthetic pathway), involves the screening of a large library of polynucleotides for individual library members which possess or encode the desired structure or functional property conferred by the polynucleotide sequence.

In a general aspect, the invention provides a sequence shuffling method, for generating libraries of recombinant polynucleotides having a desired photosynthetic carbon fixation protein characteristic which can be selected or screened for. Libraries of recombinant polynucleotides are generated from a population of related-sequence polynucleotides which comprise sequence regions which have substantial sequence identity and can be homologously recombined in vitro or in vivo. In the method, at least two species of the related-sequence polynucleotides are combined in a recombination system suitable for generating sequence-recombined polynucleotides, wherein said sequence-recombined polynucleotides comprise a portion of at least one first species of a related-sequence polynucleotide with at least one adjacent portion of at least one second species of a related-sequence polynucleotide. Recombination systems suitable for generating sequence-recombined polynucleotides can be either: (1) in vitro systems for homologous recombination or sequence shuffling via amplification or other formats described herein, or (2) in vivo systems for homologous recombination or site-specific recombination as described herein.

The population of sequence-recombined polynucleotides comprises a subpopulation of polynucleotides which possess desired or advantageous characteristics and which can be selected by a suitable selection or screening method. The selected sequence-recombined polynucleotides, which are typically relatedsequence polynucleotides, can then be subjected to at least one recursive cycle wherein at least one selected sequence-recombined polynucleotide is combined with at least one distinct species of related-sequence polynucleotide (which may itself be a selected sequence-recombined polynucleotide) in a recombination system suitable for generating sequence-recombined polynucleotides, such that additional generations of sequence-recombined polynucleotide sequences are generated from the selected sequence-recombined polynucleotides obtained by the selection or screening method employed. In this manner, recursive sequence recombination generates library members which are sequence-recombined polynucleotides possessing desired characteristics. Such characteristics can be any property or attribute capable of being selected for or detected in a screening system, and may include properties of: an encoded protein, a transcriptional element, a sequence controlling transcription, RNA processing, RNA stability, chromatin conformation, translation, or other expression property of a gene or transgene, a replicative element, a protein-binding element, or the like, such as any feature which confers a selectable or detectable property.

Nucleic acid sequence shuffling is a method for recursive in vitro or in vivo homologous or nonhomologous recombination of pools of nucleic acid fragments or polynucleotides (e.g., genes from agricultural organisms or portions thereof).

Mixtures of related nucleic acid sequences or polynucleotides are randomly or pseudorandomly fragmented, and reassembled to yield a library or mixed population of recombinant nucleic acid molecules or polynucleotides.

The present invention is directed to a method for generating a selected polynucleotide sequence (e.g., a plant rbc gene or microbe rbc gene, or combinations thereof) or population of selected polynucleotide sequences, typically in the form of amplified and/or cloned polynucleotides, whereby the selected polynucleotide sequence(s) possess a desired phenotypic characteristic of photosynthetic carbon fixation proteins can be selected for, and whereby the selected polynucleotide sequences are genetic sequences having a desired functionality and/or conferring a desired phenotypic property to an agricultural organism in which the polynucleotide has been transferred into.

In a general aspect, the invention provides a method, called “sequence shuffling,” for generating libraries of recombinant polynucleotides having a subpopopulation of library members which encode an enhanced or improved photosynthetic carbon fixation protein. Libraries of recombinant polynucleotides are generated from a population of related-sequence photosynthetic carbon fixation polynucleotides which comprise sequence regions which have substantial sequence identity and can be homologously recombined in vitro or in vivo. In the method, at least two species of the related-sequence polynucleotides are combined in a recombination system suitable for generating sequence-recombined polynucleotides, wherein said sequence-recombined polynucleotides comprise a portion of at least one first species of a related-sequence photosynthetic carbon fixation protein with at least one adjacent portion of at least one second species of a related-sequence photosynthetic carbon fixation protein. Recombination systems suitable for generating sequence-recombined polynucleotides can be either: (1) in vitro systems for homologous recombination or sequence shuffling via amplification or other formats described herein, or (2) in vivo systems for homologous recombination or site-specific recombination as described herein, or template-switching of a retroviral genome replication event. The population of sequence-recombined polynucleotides comprises a subpopulation of photosynthetic carbon fixation polynucleotide which possess desired or advantageous enzymatic characteristics and which can be selected by a suitable selection or screening method. The selected sequence-recombined photosynthetic carbon fixation polynucleotide, which are typically related-sequence polynucleotides, can then be subjected to at least one recursive cycle wherein at least one selected sequence-recombined photosynthetic carbon fixation polynucleotide is combined with at least one distinct species of related-sequence photosynthetic carbon fixation polynucleotide (which may itself be a selected sequence-recombined polynucleotide) in a recombination system suitable for generating sequencerecombined photosynthetic carbon fixation polynucleotides, such that additional generations of sequence-recombined polynucleotide sequences are generated from the selected sequence-recombined polynucleotides obtained by the selection or screening method employed. In this manner, recursive sequence recombination generates library members which are sequence-recombined polynucleotides possessing desired phenotypic or enzymatic characteristics. Such characteristics can be any property or attribute capable of being selected for or detected in a screening system.

Screening/selection produces a subpopulation of genetic sequences (or cells) expressing recombinant forms of photosynthetic carbon fixation proteins that have evolved toward acquisition of a desired enzymatic property. These recombinant forms can then be subjected to further rounds of recombination and screening/selection in any order. For example, a second round of screening/selection can be performed analogous to the first resulting in greater enrichment for genes having evolved toward acquisition of the desired enzymatic property. Optionally, the stringency of selection can be increased between rounds (e.g., if selecting for drug resistance, the concentration of drug in the media can be increased). Further rounds of recombination can also be performed by an analogous strategy to the first round generating further recombinant forms of the gene(s) or genome(s). Alternatively, further rounds of recombination can be performed by any of the other molecular breeding formats discussed. Eventually, a recombinant form of the photosynthetic carbon fixation protein is generated that has fully acquired the desired enzymatic property.

In an embodiment, the first plurality of selected library members is fragmented and homologously recombined by PCR in vitro. Fragment generation is by nuclease digestion, partial extension PCR amplification, PCR stuttering, or other suitable fragmenting means, such as described herein and in WO95/22625 published Aug. 24, 1995, and in U.S. Ser. No. 08/621,859 filed Mar. 25, 1996, PCT/US96/05480 filed Apr. 18, 1996, which are incorporated herein by reference). Stuttering is fragmentation by incomplete polymerase extension of templates. A recombination format based on very short PCR extension times can be employed to create partial PCR products, which continue to extend off a different template in the next (and subsequent) cycle(s), and effect de facto fragmentation. Template-switching and other formats which accomplish sequence shuffling between a plurality of sequence-related polynucleotides can be used. Such alternative formats will be apparent to those skilled in the art.

In an embodiment, the first plurality of selected library members is fragmented in vitro, the resultant fragments transferred into a host cell or organism and homologously recombined to form shuffled library members in vivo.

In an embodiment, the first plurality of selected library members is cloned or amplified on episomally replicable vectors, a multiplicity of said vectors is transferred into a cell and homologously recombined to form shuffled library members in vivo.

In an embodiment, the first plurality of selected library members is not fragmented, but is cloned or amplified on an episomally replicable vector as a direct repeat or indirect (or inverted) repeat, which each repeat comprising a distinct species of selected library member sequence, said vector is transferred into a cell and homologously recombined by intra-vector or inter-vector recombination to form shuffled library members in vivo.

In an embodiment, combinations of in vitro and in vivo shuffling are provided to enhance combinatorial diversity. The recombination cycles (in vitro or in vivo) can be performed in any order desired by the practitioner.

In one embodiment, the first plurality of selected library members is fragmented and homologously recombined by PCR in vitro. Fragment generation is by nuclease digestion, partial extension PCR amplification, PCR stuttering, or other suitable fragmenting means, such as described herein and in the documents incorporated herein by reference. Stuttering is fragmentation by incomplete polymerase extension of templates.

In one embodiment, the first plurality of selected library members is fragmented in vitro, the resultant fragments transferred into a host cell or organism and homologously recombined to form shuffled library members in vivo. In an aspect, the host cell is a plant cell which has been engineered to contain enhanced recombination systems, such as an enhanced system for general homologous recombination (e.g., a plant expressing a recA protein or a plant recombinase from a transgene or plant virus) or a site-specific recombination system (e.g., a cre/LOX or frt/FLP system encoded on a transgene or plant virus).

In one embodiment, the first plurality of selected library members is cloned or amplified on episomally replicable vectors, a multiplicity of said vectors is transferred into a cell and homologously recombined to form shuffled library members in vivo in a plant cell, algae cell, or bacterial cell. Other cell types may be used, if desired.

In one embodiment, the first plurality of selected library members is not fragmented, but is cloned or amplified on an episomally replicable vector as a direct repeat or indirect (or inverted) repeat, which each repeat comprising a distinct species of selected library member sequence, said vector is transferred into a cell and homologously recombined by intra-vector or inter-vector recombination to form shuffled library members in vivo in a plant cell, algae cell, or microorganism.

In an embodiment, the method employs at least one parental polynucleotide sequence that encodes a Rubisco subunit of a marine algae, such as for example and not limitation Cylindrotheca fusiformis, Olisthodiscus luteus, Cryptomonas, and Porphyridium, among others having Rubisco enzymes with a high ratio of carboxylase to oxygenase activity (Read BA and Tabita FR (1994) Arch. Biochem. Biophys. 312:210).

In an embodiment, combinations of in vitro and in vivo shuffling are provided to enhance combinatorial diversity.

At least two additional related specific formats are useful in the practice of the present invention. The first, referred to as “in silico” shuffling utilizes computer algorithms to perform “virtual” shuffling using genetic operators in a computer. As applied to the present invention, Calvin or Krebs cycle enzymes such as Rubisco nucleic acid sequence strings are recombined in a computer system and desirable products are made, e.g., by reassembly PCR or ligation of synthetic oligonucleotides, or other available techniques. In silico shuffling is described in detail in Selifonov and Stemmer in “METHODS FOR MAKING CHARACTER STRINGS, POLYNUCLEOTIDES & POLYPEPTIDES HAVING DESIRED CHARACTERISTICS” filed 02/05/1999, U.S. Ser. No. 60/118854 and “METHODS FOR MAKING CHARACTER STRINGS, POLYNUCLEOTIDES & POLYPEPTIDES HAVING DESIRED CHARACTERISTICS” by Selifonov et al. filed Oct. 12, 1999 (U.S. Ser. No. 09/416375). In brief, genetic operators (algorithms which represent given genetic events such as point mutations, recombination of two strands of homologous nucleic acids, etc.) are used to model recombinational or mutational events which can occur in one or more nucleic acid, e.g., by aligning nucleic acid sequence strings (using standard alignment software, or by manual inspection and alignment) and predicting recombinational outcomes based upon selected genetic algorithms (mutation, recombination, etc.). The predicted recombinational outcomes are used to produce corresponding molecules, e.g., by oligonucleotide synthesis and reassembly PCR. As applied to the present invention, Rubisco and other Calvin or Krebs cycle nucleic acids are aligned and recombined in silico, using any desired genetic operator, to produce character strings which are then generated synthetically for subsequent screening.

The second useful format is referred to as “oligonucleotide mediated shuffling” in which oligonucleotides corresponding to a family of related homologous nucleic acids (e.g., as applied to the present invention, families of homologous Rubisco variants of a nucleic acid) which are recombined to produce selectable nucleic acids. This format is described in detail in Crameri et al. OLIGONUCLEOTIDE MEDIATED NUCLEIC ACID RECOMBINATION filed Feb. 5, 1999, U.S. Ser. No. 60/118,813, Crameri et al. OLIGONUCLEOTIDE MEDIATED NUCLEIC ACID RECOMBINATION filed Jun. 24, 1999, U.S. Ser. No. 60/141,049; Crameri et al. OLIGONUCLEOTIDE MEDIATED NUCLEIC ACID RECOMBINATION filed Sep. 28, 1999 (U.S. Ser. No. 09/408,392, Attorney Docket Number 02-29620US); and USE OF CODON-BASED OLIGONUCLEOTIDE SYNTHESIS FOR SYNTHETIC SHUFFLING by Welch et al., filed Sep. 28, 1999 (U.S. Ser. No. 09/408,393, Attorney Docket Number 02-010070US). In brief, selected oligonucleotides corresponding to multiple homologous parental nucleic acids are synthesized, ligated and elongated (typically in a recursive format), typically either in a polymerase or ligase-mediated elongation reaction, to produce full-length photosynthetic carbon fixation polynucleotides. The technique can be used to recombine homologous or even non-homologous Rubisco nucleic acid sequences.

One advantage of oligonucleotide-mediated recombination is the ability to recombine homologous nucleic acids with low sequence similarity, or even non-homologous nucleic acids. In these low-homology oligonucleotide shuffling methods, one or more set of fragmented nucleic acids (e.g., oligonucleotides corresponding to multiple Rubisco activase encoding polynucleotides) are recombined, e.g., with a set of crossover family diversity oligonucleotides. Each of these crossover oligonucleotides have a plurality of sequence diversity domains corresponding to a plurality of sequence diversity domains from homologous or non3 homologous nucleic acids with low sequence similarity. The fragmented oligonucleotides, which are derived by comparison to one or more homologous or non-homologous nucleic acids, can hybridize to one or more region of the crossover oligos, facilitating recombination.

When recombining homologous nucleic acids, sets of overlapping family gene shuffling oligonucleotides (which are derived by comparison of homologous nucleic acids, by synthesis of corresponding oligonucleotides) are hybridized and elongated (e.g., by reassembly PCR or ligation), providing a population of recombined nucleic acids, which can be selected for a desired trait or property. The set of overlapping family shuffling gene oligonucleotides includes a plurality of oligonucleotide member types which have consensus region subsequences derived from a plurality of homologous target nucleic acids.

Typically, as applied to the present invention, family gene shuffling oligonucleotides which include one or more photosynthetic carbon fixation polynucleotides are provided by aligning homologous nucleic acid sequences to select conserved regions of sequence identity and regions of sequence diversity. A plurality of family gene shuffling oligonucleotides are synthesized (serially or in parallel) which correspond to at least one region of sequence diversity.

Sets of fragments, or subsets of fragments used in oligonucleotide shuffling approaches can be provided by cleaving one or more homologous nucleic acids (e.g., with a DNase), or, more commonly, by synthesizing a set of oligonucleotides corresponding to a plurality of regions of at least one nucleic acid (typically oligonucleotides corresponding to a full-length nucleic acid are provided as members of a set of nucleic acid fragments). In the shuffling procedures herein, these cleavage fragments can be used in conjunction with family gene shuffling oligonucleotides, e.g., in one or more recombination reaction to produce recombinant Rubisco nucleic acid(s).

One final synthetic variant worth noting is found in SHUFFLING OF CODON ALTERED GENES by Patten et al. filed Sep. 29, 1998, (U.S. Ser. No. 60/102,362), Jan. 29, 1999 (U.S. Ser. No. 60/117,729), and Sep. 28, 1999, PCT/US99/22588 (Attorney Docket Number 20-28520US/PCT). As noted in detail in this set of related applications, one way of generating diversity in a set of nucleic acids to be shuffled (i.e., as applied to the present invention, Rubisco nucleic acids), is to provide codon-altered nucleic acids which can be shuffled to provide access to sequence space not present in naturally occurring sequences. In brief, by synthesizing nucleic acids in which the codons which encode polypeptides are altered, it is possible to access a completely different mutational spectrum upon subsequent mutation of the nucleic acid. This increases the sequence diversity of the starting nucleic acids for shuffling protocols, which alters the rate and results of forced evolution procedures. Codon modification procedures can be used to modify any Rubisco nucleic acid or shuffled nucleic acid, e.g., prior to performing DNA shuffling.

In brief, oligonucleotide sets comprising codon variations are synthesized and reassembled into full-length nucleic acids. The full length nucleic acids can themselves be shuffled (e.g., where the oligonucleotides to be reassembled provide sequence diversity at selected sites), and/or the full-length sequences can be shuffled by any available procedure to produce diverse sets of photosynthetic carbon fixation polynucleotides.

### Improved Plants

Without reciting the various generalized formats of polynucleotide sequence shuffling and selection described previously or herein below, which will be referred to herein by the shorthand “shuffling,” the present invention provides methods, compositions, and uses related to creating novel or improved plants, plant cells, algal cells, soil microbes, plant pathogens, commensal microbes, or other plant-related organisms having art-recognized importance to the agricultural, horticultural, and argonomic areas (collectively, “agricultural organisms“). In particular, any plant, plant cell, algal cell, etc. can be transduced with a shuffled nucleic acid produced according to the present invention. For example, agronomically and horticulturally important plant species can be transduced. Such species include, but are not restricted to, members of the families: Graminae (including corn, rye, triticale, barley, millet, rice, wheat, oats, etc.); Leguminosae (including pea, beans, lentil, peanut, yam bean, cowpeas, velvet beans, soybean, clover, alfalfa, lupine, vetch, lotus, sweet clover, wisteria, and sweetpea); Compositae (the largest family of vascular plants, including at least 1,000 genera, including important commercial crops such as sunflower) and Rosaciae (including raspberry, apricot, almond, peach, rose, etc.), as well as nut plants (including, walnut, pecan, hazelnut, etc.) Targets for modification the evolved vectors of the invention, as well as those specified above, include plants from the genera: Agrostis, Allium, Antirrhinum, Apium, Arachis, Asparagus, Atropa, Avena (e.g., oats), Bambusa, Brassica, Bromus, Browaalia, Camellia, Cannabis, Capsicum, Cicer, Chenopodium, Chichorium, Citrus, Coffea, Coix, Cucumis, Curcubita, Cynodon, Dactylis, Datura, Daucus, Digitalis, Dioscorea, Elaeis, Eleusine, Festuca, Fragaria, Geranium, Glycine, Helianthus, Heterocallis, Hevea, Hordeum (e.g., barley), Hyoscyamus, Ipomoea, Lactuca, Lens, Lilium, Linum, Lolium, Lotus, Lycopersicon, Majorana, Malus, Mangifera, Manihot, Medicago, Nemesia, Nicotiana, Onobrychis, Oryza (e.g., rice), Panicum, Pelargonium, Pennisetum (e.g., millet), Petunia, Pisum, Phaseolus, Phleum, Poa, Prunus, Ranunculus, Raphanus, Ribes, Ricinus, Rubus, Saccharum, Salpiglossis, Secale (e.g., rye), Senecio, Setaria, Sinapis, Solanum, Sorghum, Stenotaphrum, Theobroma, Trifolium, Trigonella, Triticum (e.g., wheat), Vicia, Vigna, Vitis, Zea (e.g., corn), the Olyreae, the Pharoideae and many others.

For example, common crop plants which are targets of the present invention include corn, rice, triticale, rye, cotton, soybean, sorghum, wheat, oats, barley, millet, sunflower, canola, peas, beans, lentils, peanuts, yam beans, cowpeas, velvet beans, clover, alfalfa, lupine, vetch, lotus, sweet clover, wisteria, sweetpea and nut plants (e.g., walnut, pecan, etc).

In certain variations, naturally occurring in vivo recombination mechanisms of plants, agricultural microorganisms, or vector-host cells for intermediate replication can be used in conjunction with a collection of shuffled polynucleotide sequence variants having a desired phenotypic property to be optimized further; in this way, a natural recombination mechanism can be combined with intelligent selection of variants in an iterative manner to produce optimized variants by “forced evolution”, wherein the forced evolved variants are not expected to, nor are observed to, occur in nature, nor are predicted to occur at an appreciable frequency. The practitioner may further elect to supplement the mutational drift by introducing intentionally mutated polynucleotide species suitable for shuffling, or portions thereof, into the pool of initial polynucleotide species and/or into the plurality of selected, shuffled polynucleotide species which are to be recombined. Mutational drift may also be supplemented by the use of mutagens (e.g., chemical mutagens or mutagenic irradiation), or by employing replication conditions which enhance the mutation rate.

### Forced Evolution of Genes

The invention provides a means to evolve Rubisco (rbcS and/or rbcL)gene variants and/or suitable host cells, as well as providing a model system for evaluating a library of agents to identify candidate agents that could find use as agricultural reagents (e.g., herbicide) for commercial applications. Such agents may exhibit selectivity for inhibition of a naturally occurring Rubisco enzyme and may be substantially less effective at inhibiting a shuffled Rubisco enzyme which has been evolved to be resistant to the agent.

### Transcriptional Regulatory Sequences

Suitable transcriptional regulatory sequences include: cauliflower mosaic virus 19S and 35S promoters, NOS promoter, OCS promoter, rbcS promoter, Brassica heat shock promoter, synthetic promoters, non-plant promoters modified, if necessary, for function in plant cells, substantially any promoter that naturally occurs in a plant genome, promoters of plant viruses or Ti plasmids, tissue-preferential promoters or cis-acting elements, light-responsive promoters or cis-acting elements (e.g., rbcS LRE), hormoneresponsive cis-acting elements, developmental stage-specific promoters and cis-acting elements, viral promoters (e.g., from Tobacco Mosaic virus, Brome Mosaic Virus, Cauliflower Mosaic virus, and the like), and the like. In a variation, a transcriptional regulatory sequence from a first plant species is optimized for functionality in a second plant species by application of recursive sequence shuffling.

Transcriptional regulatory sequences for expression of shuffled rbcL sequences in chloroplasts is known in the art (Daniell et al. (1998) op.cit; O=Neill et al. (1993) The Plant Journal 3: 729; Maliga P (1993) op cit), as are homologous recombination vectors.

### Host Cells for Screening rbc Gene Shufflants

A variety of suitable host cells will be apparent to those skilled in the art. Of particular note, Form II rbcL gene shufflants can be expressed in the Cbb⁻ Rubisco deletion mutant strain of R. Rubrum and in other bacterial hosts, including E. coli, as well as higher taxonomic host cells. However, Form I subunits from higher plants are not processed correctly in bacterial host cells, so Form I rbcL and rbcS shufflants are generally expressed for Rubisco phenotype screening in Synechococcus mutants, Rubisco-deficient tobacco cells, or the like. As noted herein, a preferred host cell for the expression of eukaryotic Rubisco, particularly higher plant Rubisco, is Chlamydomonas, e.g., Chlamydomonas reinhardtii.

### Transformation

The transformation of plants and protoplasts in accordance with the invention may be carried out in essentially any of the various ways known to those skilled in the art of plant molecular biology. See, in general, Methods in Enzymology Vol. 153 (“Recombinant DNA Part D“) 1987, Wu and Grossman Eds., Academic Press, incorporated herein by reference. Additional useful general references for plant cell cloning, culture and regeneration include Jones (ed) (1995) Plant Gene Transfer and Expression Protocols—Methods in Molecular Biology, Volume 49 Humana Press Towata N.J.; Payne et al. (1992) Plant Cell and Tissue Culture in Liquid Systems John Wiley & Sons, Inc. New York, N.Y. (Payne); and Gamborg and Phillips (eds) (1995) Plant Cell, Tissue and Organ Culture; Fundamental Methods Springer Lab Manual, Springer-Verlag (Berlin Heidelberg New York) (Gamborg). A variety of cell culture media are described in Atlas and Parks (eds) The Handbook of Microbiological Media (1993) CRC Press, Boca Raton, Fla. (Atlas). Additional information for plant cell culture is found in available commercial literature such as the Life Science Research Cell Culture Catalogue (1998) from Sigma- Aldrich, Inc (St Louis, Mo.) (Sigma-LSRCCC) and, e.g., the Plant Culture Catalogue and supplement (1997) also from Sigma-Aldrich, Inc (St Louis, Mo.) (Sigma-PCCS). Additional details regarding plant cell culture are found in Croy, (ed.) (1993) Plant Molecular Biology Bios Scientific Publishers, Oxford, U.K. General texts discussing cloning and other techniques relevant to the present invention, in a variety of contexts, include: Berger and Kimmel, Guide to Molecular Cloning Techniques, Methods in Enzymology volume 152 Academic Press, Inc., San Diego, Calif. (Berger); Sambrook et al., Molecular Cloning—A Laboratory Manual (2nd Ed.), Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”)).

As used herein, the term “transformation” means alteration of the genotype of a host plant by the introduction of a nucleic acid sequence. The nucleic acid sequence need not necessarily originate from a different source, but it will, at some point, have been external to the cell into which it is to be introduced.

In one embodiment, the foreign nucleic acid is mechanically transferred by microinjection directly into plant cells by use of micropipettes. Alternatively, the foreign nucleic acid may be transferred into the plant cell by using polyethylene glycol. This forms a precipitation complex with the genetic material that is taken up by the cell (e.g., by incubation of protoplasts with “naked DNA” in the presence of polyethylenelycol)(Paszkowski et al., (1984) EMBO J. 3:2717-22; Baker et al (1985) Plant Genetics, 201-211; Li et al. (1990) Plant Molecular Biology Report 8(4)276-291].

In another embodiment of this invention, the introduced gene may be introduced into the plant cells by electroporation (Fromm et al., (1985) “Expression of Genes Transferred into Monocot and Dicot Plant Cells by Electroporation,” Proc. Natl Acad. Sci. USA 82:5824, which is incorporated herein by reference). In this technique, plant protoplasts are electroporated in the presence of plasmids or nucleic acids containing the relevant genetic construct. Electrical impulses of high field strength reversibly permeabilize biomembranes allowing the introduction of the plasmids. Electroporated plant protoplasts reform the cell wall, divide, and form a plant callus. Selection of the transformed plant cells with the transformed gene can be accomplished using phenotypic markers.

Cauliflower mosaic virus (CaMV) may also be used as a vector for introducing the foreign nucleic acid into plant cells (Hohn et al., (1982) “Molecular Biology of Plant Tumors,” Academic Press, New York, pp.549-560; Howell, U.S. Pat. No. 4,407,956). CaMV viral DNA genome is inserted into a parent bacterial plasmid creating a recombinant DNA molecule which can be propagated in bacteria. After cloning, the recombinant plasmid again may be cloned and further modified by introduction of the desired DNA sequence into the unique restriction site of the linker. The modified viral portion of the recombinant plasmid is then excised from the parent bacterial plasmid, and used to inoculate the plant cells or plants.

Another method of introduction of nucleic acid segments is high velocity ballistic penetration by small particles with the nucleic acid either within the matrix of small beads or particles, or on the surface (Klein et al., (1987) Nature 327:70-73). Although typically only a single introduction of a new nucleic acid segment is required, this method particularly provides for multiple introductions.

A method of introducing the nucleic acid segments into plant cells is to infect a plant cell, an explant, a meristem or a seed with Agrobacterium tumefaciens transformed with the segment. Under appropriate conditions known in the art, the transformed plant cells are grown to form shoots, roots, and develop further into plants. The nucleic acid segments can be introduced into appropriate plant cells, for example, by means of the Ti plasmid of Agrobacterium tumefaciens. The Ti plasmid is transmitted to plant cells upon infection by Agrobacterium tumefaciens, and is stably integrated into the plant genome (Horsch et al., (1984) “Inheritance of Functional Foreign Genes in Plants,” Science, 233:496-498; Fraley et al., (1983) Proc. Natl. Acad. Sci. USA 80:4803).

Ti plasmids contain two regions essential for the production of transformed cells. One of these, named transfer DNA (T DNA), induces tumor formation. The other, termed virulent region, is essential for the introduction of the T DNA into plants. The transfer DNA region, which transfers to the plant genome, can be increased in size by the insertion of the foreign nucleic acid sequence without its transferring ability being affected. By removing the tumor-causing genes so that they no longer interfere, the modified Ti plasmid can then be used as a vector for the transfer of the gene constructs of the invention into an appropriate plant cell, such being a “disabled Ti vector.”

All plant cells which can be transformed by Agrobacterium and whole plants regenerated from the transformed cells can also be transformed according to the invention so as to produce transformed whole plants which contain the transferred foreign nucleic acid sequence.

There are presently at least three different ways to transform plant cells with Agrobacterium: (1) co-cultivation of Agrobacterium with cultured isolated protoplasts; (2) transformation of cells or tissues with Agrobacterium, or (3) transformation of seeds, apices or meristems with Agrobacterium.

Method (1) uses an established culture system that allows culturing protoplasts and plant regeneration from cultured protoplasts.

Method (2) implies (a) that the plant cells or tissues can be transformed by Agrobacterium and (b) that the transformed cells or tissues can be induced to regenerate into whole plants.

Method (3) uses micropropagation. In the binary system, to have infection, two plasmids are needed: a T-DNA containing plasmid and a vir plasmid. Any one of a number of T-DNA containing plasmids can be used, the main issue being that one be able to select independently for each of the two plasmids.

After transformation of the plant cell or plant, those plant cells or plants transformed by the Ti plasmid so that the desired DNA segment is integrated can be selected by an appropriate phenotypic marker. These phenotypic markers include, but are not limited to, antibiotic resistance, herbicide resistance or visual observation. Other phenotypic markers are known in the art and may be used in this invention.

A suitable host for screeining Rubisco activase variants, e.g., shufflants, is E. coli. Another suitable host is a species of Chlamydomonas, preferably Chlamydomonas reindartii.

In another preferred embodiment of the invention a species of Chlamydomonas, preferably Chlamydomonas reindartii, is engineered to make it a better host for expression and screening of higher plant rubisco. Chlamydomonas is a eukaryotic unicellular green algae that is often regarded as a model system for higher plant cells (Weeks, 1992, The Plant Cell). Like the enzyme from higher plants, the Chlamydomonas Rubisco large subunit (L) is encoded by a chloroplast gene (rbcL) and its small subunit (S) is encoded by a nuclear gene (rbcS) family. Knock-out mutants of both rbcL and rbcS have been demonstrated in Chlamydomonas (Newman et al, 1991, Mol. Gen. Genet. 230, 65-74; Khrebtukova and Spreitzer, 1996, PNAS, 93:13689-13693). Chlamydomonas L shares ˜90% amino acid sequence identity with higher plant L. Higher plant (non-solanaceae) Rubisco can be activated by Chlamydomonas activase and vice versa (Wang et al, 1992, Plant Physiol. 100, 1858-1862); Rubisco-deficient Chlamydomonas strains can be maintained by heterotrophic growth on medium containing acetate. Functional Rubisco transformants (shuffled variants) can be selected simply by their ability to confer autotrophic growth.

With the emergence of chloroplast transformation techniques, it has become practical to express in vitro-mutagenized rbcL in the chloroplasts of the green alga, Chlamydomonas reinhardtii (Zhu G, Spreitzer (1994) Directed mutagenesis of chloroplast ribulose-bisphosphate carboxylase/oxygenase: substitutions at large subunit asparagine 123 and serine 379 decrease CO₂/O₂ specificity. J. Biol. Chem. 269: 3952-3956.). A rbcL-deletion mutant can be created by homologous recombination. In one aspect of the invention a higher plant rbcL encoding polynucleotide is introduced into a Chlamydomonas rbcL-deletion mutant.

In one aspect, higher plant L subunit is expressed and assembled with Chlamydomonas Rubisco S, whereby a functional Rubisco is formed and selected by its ability to confer a photoautotrophic phenotype. Because the residues that form the active site are solely from Rubisco L and because research suggests that the L plays a major role in determination of CO₂/O₂ selectivity, improvement of CO₂ selectivity may be achieved by shuffling Rubisco L alone. In this case, a Rubisco L-deficient mutant is all that will be required.

However, in the case where a higher plant L and Chlamydomonas S are unable to associate into a functional holoenzyme, it may be desirable to create mutant strains in which small subunit genes or both large and small subunit genes are deleted or replaced. In preferred embodiments of the invention, deletion or replacement is accomplished using a combination of fast neutron bombardment and selection by preferential amplification, as described in U.S. patent application No. 09/285,512 and Li, X, Song, Y, Century, K, Striaght, S, Ronald, P, Dong, X, Lassner, M, Zhang, Y (2001) A fast neutron deletion mutagenesis-based reverse genetics system for plants. Plant Journal (In Press).

For example, if Chlamydomonas Rubisco small subunit is incompatible with higher plant large subunit, a higher plant small subunit gene (rbcS) can be deposited in a Chlamydomonas host where its native rbcS genes have been knocked out. The Chlamydomonas strain with higher plant Rubisco S can then be used to host shuffled higher plant Rubisco L. The strain would have native rbcL (if it is shown that the native L protein cannot functionally interact with higher plant S subunit). An alternative approach would employ is a strain that contains no Rubisco genes, in which one can directly introduce higher plant Rubisco L and S into the Chlamydomonas chloroplast. In this case, rbcL and rbcS can be shuffled simultaneously.

Because the small subunit is coded by a nuclear gene family, it is difficult to knock out all rbcS genes in a nuclear genome. An advantage of using Chlamydomonas as a host system is that the elimination of both rbcS1 and rbcS2 genes has been demonstrated by a random insertional mutagenesis (Khrebtukova and Spreitzer, 1996, PNAS, 93:13689-13693). It has been shown that a Rubisco small subunit deficient strain can be maintained in acetate medium.

There are three possible alternative mutant strains that can be employed if higher plant rbcL is unable to complement the L-deficient strain of Chlamydomonas. The first of these would be one in which the S-deficient strain mentioned above is transformed with rbcS from higher plants. If this strain is still photosynthesis-deficient, it could be used to host a library of variants of higher plant rbcL, with photoautotrophy serving as a selection for functional Rubisco.

In the unlikely event that the strain is already photoautotrophic due to a compatible interaction between the native L with the introduced higher plant S (unlikely at this stage, because we already would have found that higher plant L cannot interact with native S), it would be necessary to further modify this strain by deleting the endogenous rbcL gene. Thus, the second alternative strain would be deficient in rbcL, have higher plant rbcS, and would serve to host a library of higher plant rbcL variants.

The third alternative strain that may be of use is one which is deficient in both large and small subunit genes. This would be used to create a tandem or operon construct of both genes, so that both could be shuffled simultaneously. A preferred starting material for this strain would be a small subunit-deficient strain, which could be further modifedy by deleting the rbcL gene by homologous recombination.

Once the appropriate Rubisco L and S deficient mutant strains are created, wild type higher plant Rubisco genes (rbcL and rbcS) can be introduced into these hosts to test their compatibility to express and assemble higher plant Rubisco. Two different experiments can be conducted: 1) introduce rbcL and rbcS separately into chloroplast and nuclear genomes; 2) introduce both rbcL and rbcS into the chloroplast in a tandem construct.

Expression of a tobacco rbcS in tobacco chloroplasts has been demonstrated and the functional holoenzyme was obtained (Whitney and Andrews, 2001, The Plant Cell, 13:193-205). Although it was seen that the expression level of 10 rbcS is quite low in chloroplasts, the poor expression could be due to differences in codon usage between nuclear and chloroplastic genomes. Therefore, for successful expression of higher plant Rubisco in Chlamydomonas chloroplasts, it is desirable to optimize the codon usage of higher plant Rubisco for expression in the Chlamydomonas chloroplast. Optimization of codon usage is described, for example, in U.S. provisional application 60/227,719, filed Aug. 24, 2000.

In general, codon optimization can be used to improve expression of higher plant derived Rubisco in chlamydomonas, whether the heterologous construct is introduced into the nuclear or chloroplast genome. For example, a codon of a higher plant Rubisco encoding polynucleotide can be replaced with synonomous codon that is preferentially used in the targeted Chlamydomonas genome. Alternatively, by the replacement of one or more synonomous codons the GC content of the coding sequence can be altered to render it closer to the typical GC content of a naturally occurring chlamydomonas ORF of the targetted genome. For example, the GC content of a typical Arabidopsis thaliana nuclear genomic ORF is much lower than a typical Chlamydomonas nuclear genomic ORF, particularly at the 3^{rd} base position of the codons. Expression of Rubisco small subunit derived from Arabidopsis in Chlamydomonas can be improved by increasing the GC content, particularly at 3^{rd} base positions.

Another method for improving the expression of higher plant-derived Rubisco is to alter intron usage of the coding sequence. Examples of intron modifications that can improve expression of rubsico in heterologous host systems include removal of introns, modification of intron sequences, and replacement of naturally occurring intron sequences. For example, in some cases expression can be improved by removal of one or more naturally occurring introns. Alternatively, expression can be improved by replacing a naturally occuring intron with another intron. Replacement of a naturally occurring intron with an intron derived from a gene native to the host system can improve expression. For example, to improve expression of a higher plant rubisco in chlamydomonas it can be beneficial to replace an intron in the rubisco coding sequence with an intron derived from a gene naturally expressed in chiamydomonas. A naturally occurring intron can be modified for better expression in a host system of interest, e.g., by altering the intron sequence to more closely resemble an intron sequence that is effectively processed by the host system. Introns can be modified, deleted or replaced using methods that are well known in the art.

Once the Chlamydomonas expression system is established, the functional mutant variants can be selected by photoautotrophic growth. The major limitation in using Chlamydomonas as a host system in a screening-intensive approach such as DNA shuffling is its low transformation efficiency (approximately 10-5 to 10-6 transformants/ug). With a substantial increase in chloroplast transformation efficiency, or by repeated transformation with shuffled library DNA (amplified in E. coli) to generate a reasonably large population of transformants, this system will be adequate for primary screening of shuffled libraries.

### Rubisco Activase Variant Library Screening

Because Rubisco activase has an ATP hydrolysis activity that is associated with its ability to activate Rubisco, a high throughput screening system can be used based on its ATP hydrolysis activity, wherein the Pi released is measured using the Malachite green assay. For example, to screen for variants with enhanced thermal stability, a collection of E. coli host cells harboring Rubisco activase variants are induced to express Rubisco activase and are incubated at 50° C. for 1 hr. The cells can be collected by centrifugation, and resuspended in buffer containing ATP for the ATPase assay. In some embodiments of the invention the Rubisco activase can be purified or partially purified prior to determination of ATPase activity. Methods for determining ATPase activity of Rubisco activase are known in the art, see, e.g., Crafts-Brandner and Salvucci, 2000.

Once the thermostable mutant enzymes are identified, a further test for their ability to activate Rubisco under high temperature and their compatibility with shuffled Rubisco can be performed by a low-throughput Rubisco activation assay.

### Gene Replacement

In some embodiments of the invention, one or more endogenous photosynthetic carbon fixation proteins of an organism such as a plant are replaced with an enhanced variant as described herein. In preferred embodiments of the invention, the corresponding endogenous gene is rendered non-functional in the transgenic organism, i.e., one or more of the corresponding genes are “knocked-out.“A preferred method for accomplishing targeted deletion or replacement is accomplished using a combination of fast neutron bombardment and selection by preferential amplification, as described in U.S. patent application No. 09/285,512 and in Li, X, Song, Y, Century, K, Striaght, S, Ronald, P, Dong, X, Lassner, M, Zhang, Y (2001) A fast neutron deletion mutagenesis-based reverse genetics system for plants. Plant Journal (27:235-242). For example, this methodology can be used to create an Arabidopsis line with three tandem rbcS genes deleted. Although this is a lethal mutation, the line can be maintained in a heterozygous state. After introduction of a gene improved by DNA shuffling, one can segregate out the endogenous genes. A similar approach can be used to replace the endogenous Rubisco activase genes with improved versions of the activase.

### Protoplast Transformation

Numerous protocols for establishment of transformable protoplasts from a variety of plant types and subsequent transformation of the cultured protoplasts are available in the art and are incorporated herein by general reference. For examples, see Hashimoto et al. (1990) Plant Physiol. 93: 857; Plant Protoplasts, Fowke LC and Constabel F, eds., CRC Press (1994); Saunders et al. (1993) Applications of Plant hi Vitro Technology Symposium, UPM, Nov. 16-18, 1993; and Lyznik et al. (1991) BioTechniques 10: 295, each of which is incorporated herein by reference). All plants from which protoplasts can be isolated and cultured to give whole regenerated plants can be transformed by the present invention so that whole plants are recovered which c6ntain the transferred foreign gene. Some suitable plants include, for example, species from the genera Fragaria, Lotus, Medicago, Onobrychis, Trifolium, Trigonella, Vigna, Citrus, Linum, Geranium, Manihot, Daucus, Arabidopsis, Brassica, Raphanus, Sinapis, Atropa, Capsicum, Hyoscyamus, Lycopersicon, Nicotiana, Solanum, Petunia, Digitalis, Majorana, Ciohorium, Helianthus, Lactuca, Bromus, Asparagus, Antirrhinum, Hererocallis, Nemesia, Pelargonium, Panicum, Pennisetum, Ranunculus, Senecio, Salpiglossis, Cucumis, Browaalia, Glycine, Lolium, Zea, Triticum, Sorghum, and Datura.

It is known that practically all plants can be regenerated from cultured cells or tissues, including but not limited to all major cereal crop species, sugarcane, sugar beet, cotton, fruit and other trees, legumes and vegetables. Limited knowledge presently exists on whether all of these plants can be transformed by Agrobacterium. Species which are a natural plant host for Agrobacterium may be transformable in vitro. Although monocotyledonous plants, and in particular, cereals and grasses, are not natural hosts to Agrobacterium, work to transform them using Agrobacterium has also been successfully carried out by numerous investigators (Hooykas-Van Slogteren et al., (1984) Nature 311:763-764; Hernalsteens et al., (1984) EMBO J. 3:3039-41; Byteiber, et al. (1987) Proc. Natl. Acad. Sci. USA: 5345-5349; Graves and Goldman, (1986) Plant Mol. Biol 7: 43-50; Grimsley et al. (1988) Biochemistry 6: 185-189; WO 86/03776; Shimamoto et al. Nature (1989) 338: 274-276). Monocots may also be transformed by techniques or with vectors other than Agrobacterium. For example, monocots have been transformed by electroporation (Fromm et al. [1986] Nature 319:791-793; Rhodes et al. Science [1988] 240: 204-207), direct gene transfer (Baker et al. [1985] Plant Genetics 201-211), by using pollen-mediated vectors (EP 0 270 356), and by injection of DNA into floral tillers (de la Pena et al. [1987], Nature 325:274-276). Additional plant genera that may be transformed by Agrobacterium include Chrysanthemum, Dianthus, Gerbera, Euphorbia, Pelaronium, Ipomoea, Passiflora, Cyclamen, Malus, Prunus, Rosa, Rubus, Populus, Santalum, Allium, Lilium, Narcissus, Ananas, Arachis, Phaseolus and Pisum. Chloroplast Transformation

As the rbcL gene of higher plants is encoded on the chloroplast genome and expressed in chloroplasts, it is generally useful to transform the shufflant Form I rbcL encoding sequences into chloroplasts if the host cells are derived from higher plants. Numerous methods are available in the art to accomplish the chloroplast transformation and expression (Daniell et al. (1998) op.cit; O'Neill et al. (1993) The Plant Journal 3: 729; Maliga P (1993) op cit). The rbcL expression construct comprises a transcriptional regulatory sequence functional in plants operably linked to a polynucleotide encoding an enhanced Rubisco protein subunit. With respect to polynucleotide sequences encoding Form I Rubisco L subunit proteins, it is generally desirable to express such encoding sequences in plastids, such as chloroplasts, for appropriate transcription, translation, and processing. With reference to expression cassettes which are designed to function in chloroplasts, such as an expression cassette encoding a large subunit of Rubisco (rbcL) in a higher plant, the expression cassette comprises the sequences necessary to ensure expression in chloroplasts 15 typically the Rubisco L subunit encoding sequence is flanked by two regions of homology to the plastid genome so as to effect a homologous recombination with the chloroplastid genome; often a selectable marker gene is also present within the flanking plastid DNA sequences to facilitate selection of genetically stable transformed chloroplasts in the resultant transplastonic plant cells (see Maliga P (1993) TIBTECH 11: 101; Daniell et al. (1998) Nature Biotechnology 16: 346, and references cited therein).

### Recovery of Selected Polynucleotide Sequences

A variety of selection and screening methods will be apparent to those skilled in the art, and will depend upon the particular phenotypic properties that are desired. The selected shuffled genetic sequences can be recovered for further shuffling or for direct use by any applicable method, including but not limited to: recovery of DNA, RNA, or cDNA from cells (or PCR-amplified copies thereof) from cells or medium, recovery of sequences from host chromosomal DNA or PCR-amplified copies thereof, recovery of episome (e.g., expression vector) such as a plasmid, cosmid, viral vector, artificial chromosome, and the like, or other suitable recovery method known in the art.

Any suitable art-known method, including RT-PCR or PCR, can be used to obtain the selected shufflant sequence(s) for subsequent manipulation and shuffling.

### Backcrossing

After a desired photosynthetic carbon fixation protein phenotype is acquired to a satisfactory extent by a selected shuffled gene or portion thereof, it is often desirable to remove mutations which are not essential or substantially important to retention of the desired phenotype (superfluous mutations). For example, this is particularly desirable when the shuffled gene sequence is to be reintroduced back into a higher plant, as it is often preferred to harmonize the shufflant Rubisco subunit sequence with the endogenous Rubisco subunit sequence in the higher plant taxonomic species genome while retaining the desired Rubisco pheotype obtained from the iterative shuffling/selection process. Superfluous mutations can be removed by backcrossing, which is shuffling the selected shuffled rbcL gene(s) with one or more parental rbcL gene and/or naturally-occurring rbcL gene(s) (or portions thereof) and selecting the resultant collection of shufflants for those species that retain the desired phenotype. The same process may be employed for the rbcS genes. By employing this method, typically in two or more recursive cycles of shuffling against parental or naturally-occurring viral genome(s) (or portions thereof) and selection for retention of the desired Rubisco phenotype, it is possible to generate and isolate selected shufflants which incorporate substantially only those mutations necessary to confer the desired phenotype, whilst having the remainder of the genome (or portion thereof) consist of sequence which is substantially identical to the parental (or wildtype) sequence(s). As one example of backcrossing, a pea Rubisco subunit gene (small subunit) can be shuffled and selected for the capacity to substantially function in any Angiosperm plant cells; the resultant selected shufflants can be backcrossed with one or more Rubisco genes of a particular plant species and selected for the capacity to retain the capacity to confer the phenotype. After several cycles of such backcrossing, the backcrossing will yield gene(s) which contain the mutations necessary for the desired phenotype, and will otherwise have a genomic sequence substantially identical to the genome(s) of the host genome.

Isolated components (e.g., genes, regulatory sequences, replication origins, and the like) can be optimized and then backcrossed with parental sequences so as to obtain optimized components which are substantially free of superfluous mutations.

### Transgenic Hosts

Transgenes and expression vectors to express shufflant rbc sequences can be constructed by any suitable method known in the art; by either PCR or RT-PCR amplification from a suitable cell type or by ligating or amplifying a set of overlapping synthetic oligonucleotides; publicly available sequence databases and the literature can be used to select the polynucleotide sequence(s) to encode the specific protein desired, including any mutations, consensus sequence, or mutation kernal desired by the practitioner. The coding sequence(s) are operably linked to a transcriptional regulatory sequence and, if desired, an origin of replication. Antisense or sense-suppression transgenes and genetic sequences can be optimized or adapted for particular host cells and organisms by the described methods.

The transgene(s) and/or expression vectors are transferred into host cells, protoplasts, pluripotent embryonic plant cells, microbes, or fungi by a suitable method, such as for example lipofection, electroporation, microinjection, biolistics, Agrobacterium tumefaciens transduction of Ti plasmid, calcium phosphate precipitation, PEG-mediated DNA uptake, electroporation, electrofusion, or other method. Stable transfectant host cells can be prepared by art-known methods, as can transgenic cell lines.

### Target Plants

As used herein, “plant” refers to either a whole plant, a plant part, a plant cell, or a group of plant cells. The class of plants which can be used in the method of the invention is generally as broad as the class of higher plants amenable to protoplast transformation techniques, including both monocotyledonous and dicotyledonous plants. It includes plants of a variety of ploidy levels, including polyploid, diploid and haploid, and may employ non-regenerable cells for certain aspects which do not require development of an adult plant for selection or in vivo shuffling.

As noted, preferred plants for the transformation and expression of Rubisco include agronomically and horticulturally important species. Such species include, but are not restricted to members of the families: Graminae (including corn, rye, triticale, barley, millet, rice, wheat, oats, etc.); Leguminosae (including pea, beans, lentil, peanut, yam bean, cowpeas, velvet beans, soybean, clover, alfalfa, lupine, vetch, lotus, sweet clover, wisteria, and sweetpea); Compositae (the largest family of vascular plants, including at least 1,000 genera, including important commercial crops such as sunflower) and Rosaciae (including raspberry, apricot, almond, peach, rose, etc.), as well as nut plants (including, walnut, pecan, hazelnut, etc.).

Targets for the invention also include plants from the genera: Agrostis, Allium, Antirrhinum, Apium, Arachis, Asparagus, Atropa, Avena (e.g., oats), Bambusa, Brassica, Bromus, Browaalia, Camellia, Cannabis, Capsicum, Cicer, Chenopodium, Chichorium, Citrus, Coffea, Coix, Cucumis, Curcubita, Cynodon, Dactylis, Datura, Daucus, Digitalis, Dioscorea, Elaeis, Eleusine, Festuca, Fragaria, Geranium, Glycine, Helianthus, Heterocallis, Hevea, Hordeum (e.g., barley), Hyoscyamus, Ipomoea, Lactuca, Lens, Lilium, Linum, Lolium, Lotus, Lycopersicon, Majorana, Malus, Mangifera, Manihot, Medicago, Nemesia, Nicotiana, Onobrychis, Oryza (e.g., rice), Panicum, Pelargonium, Pennisetum (e.g., millet), Petunia, Pisum, Phaseolus, Phleum, Poa, Prunus, Ranunculus, Raphanus, Ribes, Ricinus, Rubus, Saccharum, Salpiglossis, Secale (e.g., rye), Senecio, Setaria, Sinapis, Solanum, Sorghum, Stenotaphrum, Theobroma, Trifolium, Trigonella, Triticum (e.g., wheat), Vicia, Vigna, Vitis, Zea (e.g., corn), and the Olyreae, the Pharoideae and many others. Common crop plants which are targets of the present invention include corn, rice, triticale, rye, cotton, soybean, sorghum, wheat, oats, barley, millet, sunflower, canola, peas, beans, lentils, peanuts, yam beans, cowpeas, velvet beans, clover, alfalfa, lupine, vetch, lotus, sweet clover, wisteria, sweetpea and nut plants (e.g., walnut, pecan, etc).

### Regeneration

Normally, regeneration will be involved in obtaining a whole plant from the transformation process. The term “transgenote” refers to the immediate product of the transformation process and to resultant whole transgenic plants.

The term “regeneration” as used herein, means growing a whole plant from a plant cell, a group of plant cells, a plant part or a plant piece (e.g. from a protoplast, callus, or tissue part).

Plant regeneration from cultural protoplasts is described in Evans et al., “Protoplasts Isolation and Culture,” Handbook of Plant Cell Cultures 1:124-176 (MacMillan Publishing Co. New York 1983); M. R. Davey, “Recent Developments in the Culture and Regeneration of Plant Protoplasts,” Protoplasts, (1983)—Lecture Proceedings, pp.12-29, (Birkhauser, Basal 1983); P. J. Dale, “Protoplast Culture and Plant Regeneration of Cereals and Other Recalcitrant Crops,” Protoplasts (1983)—Lecture Proceedings, pp. 31-41, (Birkhauser, Basel 1983); and H. Binding, “Regeneration of Plants,” Plant Protoplasts, pp.21-73, (CRC Press, Boca Raton 1985).

Additional details regarding plant regeneration are found in Jones (ed) (1995) Plant Gene Transfer and Expression Protocols—Methods in Molecular Biology, Volume 49 Humana Press Towata N.J.; Payne et al. (1992) Plant Cell and Tissue Culture in Liquid Systems John Wiley & Sons, Inc. New York, N.Y. (Payne); Gamborg and Phillips (eds) (1995) Plant Cell Tissue and Organ Culture; Fundamental Methods Springer Lab Manual, Springer-Verlag (Berlin Heidelberg New York) (Gamborg) and in Croy, (ed.) (1993) Plant Molecular Biology.

Regeneration from protoplasts varies from species to species of plants, but generally a suspension of transformed protoplasts containing copies of the exogenous sequence is first made. In certain species embryo formation can then be induced from the protoplast suspension, to the stage of ripening and germination as natural embryos. The culture media will generally contain various amino acids and hormones, such as auxin and cytokinins. It is sometimes advantageous to add glutamic acid and proline to the medium, especially for such species as corn and alfalfa. Shoots and roots normally develop simultaneously. Efficient regeneration will depend on the medium, on the genotype, and on the history of the culture. If these three variables are controlled, then regeneration is fully reproducible and repeatable.

Regeneration also occurs from plant callus, explants, organs or parts. Transformation can be performed in the context of organ or plant part regeneration. See, Methods in Enzymology, supra; also Methods in Enzymology, Vol. 118; and Klee et al., (1987) Annual Review of Plant Physiology, 38:467-486.

In vegetatively propagated crops, the mature transgenic plants are propagated by the taking of cuttings or by tissue culture techniques to produce multiple identical plants for trialling, such as testing for production characteristics. Selection of desirable transgenotes is made and new varieties are obtained thereby, and propagated vegetatively for commercial sale.

In seed propagated crops, the mature transgenic plants are self crossed to produce a homozygous inbred plant. The inbred plant produces seed containing the gene for the newly introduced foreign gene activity level. These seeds can be grown to produce plants that would produce the selected phenotype.

The inbreds according to this invention can be used to develop new hybrids. In this method a selected inbred line is crossed with another inbred line to produce the hybrid. The offspring resulting from the first experimental crossing of two parents is known in the art as the F1 hybrid, or first filial generation. Of the two parents crossed to produce F1 progeny according to the present invention, one or both parents can be transgenic plants.

Parts obtained from the regenerated plant, such as flowers, seeds, leaves, branches, fruit, and the like are covered by the invention, provided that these parts comprise cells which have been so transformed. Progeny and variants, and mutants of the regenerated plants are also included within the scope of this invention, provided that these parts comprise the introduced DNA sequences. Progeny and variants, and mutants of the regenerated plants are also included within the scope of this invention.

While the foregoing invention has been described in some detail for purposes of clarity and understanding, it will be clear to one skilled in the art from a reading of this disclosure that various changes in form and detail can be made without departing from the true scope of the invention. For example, all the techniques and, compositions described above may be used in various combinations. All publications, patent documents (e.g., applications, patents, etc.) or other references cited in this application are incorporated by reference in their entirety for all purposes to the same extent as if each individual publication or patent document were individually so denoted.

What is claimed is:

1. A method for obtaining an isolated polynucleotide comprising a sequence encoding a protein having Rubisco activase activity, the method comprising:
recombining a plurality of parental polynucleotide species encoding at least one protein having Rubisco activase activity under conditions suitable for sequence shuffling to form a resultant library of sequence-shuffled polynucleotides;
transferring said library into a plurality of host cells, thereby forming a library of transformants wherein sequence-shuffled Rubisco activase polynucleotides are expressed;
identifying at least one transformant from said library that expresses a protein having a Rubisco activase activity that is significantly enhanced relative to the Rubisco activase activity of proteins encoded by the plurality of parental polynucleotide species, wherein the identified transformant contains a polynucleotide comprising a sequence encoding the protein having an enhanced Rubisco activase activity; thereby obtaining a polynucleotide comprising a sequence encoding the protein having an enhanced Rubisco acctivase activity.

2. The method of claim 1, wherein the encoded protein having an enhanced Rubisco activase activity has improved temperature stability relative to proteins encoded by the plurality of polynucleotide species.

3. The method of claim 1, wherein the encoded protein having an enhanced Rubisco activase activity has an higher temperature of optimum activity relative to proteins encoded by the plurality of polynucleotide species.

4. The method of claim 1, wherein the encoded protein having an enhanced Rubisco activase activity has significantly greater Rubisco activase activity relative to proteins encoded by the plurality of polynucleotide species.

5. The method of claim 1, wherein the encoded protein having an enhanced Rubisco activase activity has significantly greater ATPase activity relative to proteins encoded by the plurality of polynucleotide species.

6. The method of claim 1, wherein the step of identifying at least one transformant from said library that expresses a protein having a significantly enhanced.Rubisco activase activity comprises assaying for ATPase activity.

7. A method for expressing an enzyme having rubisco activity, the method comprising:
introducing a polynucleotide encoding an enzyme having rubisco activity into a chlamydomonas cell; and
culturing the chlamydomonas cell under conditions where the enyzme is expressed.

8. The method of claim 7, wherein the enzyme having rubisco activity is a Rubisco Form I L subunit.

9. The method of claim 7, wherein the enzyme having rubisco activity is a Rubisco Form I S subunit.

10. The method of claim 7, wherein the enzyme having rubisco activity is a Rubisco Form II subunit.

11. The method of claim 7, wherein the polynucleotide encoding an enzyme having rubisco activity is introduced into a nuclear genome of the chlamydomonas cell.

12. The method of claim 7, wherein the polynucleotide encoding an enzyme having rubisco activity is introduced into a chloroplast genome of the chlamydomonas cell.

13. The method of claim 11, comprising replacing a codon in the polynucleotide with a synonomous codon prior to introducing the polynucleotide into the chlamydomonas cell, wherein the codon replacement renders the codon usage of the polynucleotide more compatible with the chlamydomonas nuclear genome.

14. The method of claim 12, comprising replacing a codon in the polynucleotide with a synonomous codon prior to introducing the polynucleotide into the chlamydomonas cell, wherein the codon replacement renders the codon usage of the polynucleotide more compatible with the chlamydomonas chloroplast genome.

15. The method of claim 7, comprising modifying the intron usage in the polynucleotide prior to introducing the polynucleotide into the chlamydomonas cell.

16. The method of claim 15, wherein the intron usage in the polynucleotide is modified by removing an intron.

17. The method of claim 15, wherein the intron usage in the polynucleotide is modified by replacing an intron in the polynucleotide with an intron that occurs naturally in a chlamydomonas gene.

18. The method of claim 7, wherein the polynucleotide encodes a rubisco subunit derived from a eukaryote.

19. The method of claim 18, wherein the polynucleotide encodes a rubisco subunit derived from a higher plant.

20. The method of claim 19, wherein the polynucleotide encodes a shuffled variant of a rubisco subunit derived from a higher plant.
